(19) United States
(12) Patent Application Publication
(10) Pub. No.: US 2007/0003997 A1
(43) Pub. Date: Jan. 4, 2007

(54) Method and apparatus for detecting bacteria

(75) Inventors: Yukio Kemmochi, Yokohama-shi (JP); Kaori Tsutsumi, Ohta-ku (JP); Kensuke Onda, Ohta-ku (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: EBARA CORPORATION, Ohta-ku (JP)

(21) Appl. No.: 11/428,046

(22) Filed: Jun. 30, 2006

(30) Foreign Application Priority Data
Jul. 1, 2005 (JP) 2005-193510
Jun. 19, 2006 (JP) 2006-168893

(51) Int. Cl. C12Q 1/04

(52) U.S. Cl. 435/34

(57) ABSTRACT

A method and an apparatus for detecting/quantifying bacteria in a sample rapidly and simply with sufficient sensitivity and accuracy by an enzyme activity method are provided. A test sample fluid is passed through a filter membrane 11 having a pore diameter of 0.6 to 5.0 μm and/or a flow rate of distilled water passed of 50 to 500 mL/min·cm² to collect the bacteria in the sample fluid on the filter membrane 11, and a lysing agent and an enzyme reaction substrate fluid are added to the bacteria collected on the filter membrane 11 to allow enzyme substrate reaction to proceed, and the enzyme activity is measured by a measuring device 33 to quantify the number of the bacteria in the sample fluid.

### BACKGROUND OF THE INVENTION

1. Technical Field

The present invention relates to a method and an apparatus for detecting/quantifying bacteria in a sample. Particularly, the present invention is useful as a method of detecting/quantifying E. coli, coliforms and the like in sample water, rapidly and simply, with high sensitivity.

2. Background Art

Escherichia coli (hereinafter also referred to as E. coli) is important as an indicator of fecal pollution. Coliforms are also important as hygienic indicators of fecal pollution and, simultaneously, are used an indicator for confirming the effect of disinfection.

Coliforms are defined as aerobic and facultative anaerobic, Gram-negative, non-spore-forming rod-shaped bacteria and ferment lactose with gas and acid formation within 48 hours at 35° C. in lactose bouillon medium, and are mainly constituted of Escherichia genus, Citrobacter genus, Enterobacter genus and Klebsiella genus. Since the coliforms have characteristic features such that they are present when pathogenic bacteria are present; they neither increase nor decrease in the hydrosphere; they are present more than pathogenic bacteria; they are more resistant to disinfectant than pathogenic bacteria; they easily proliferate on a simple culture medium and can be reliably confirmed; they are randomly dispersed in a sample to be detected; they are not inhibited to proliferate by other bacteria on seeding on a medium, and so on, coliforms have been utilized for a long time as an indicator of pollution of fecal pathogenic bacteria (enteropathogenic bacteria). However, the coliforms contain bacteria originating from nature, such as those found in soil in addition to fecal pathogenic bacteria, and thus the detection of the coliforms does not identify the bacteria alone derived from the feces of warm-blooded animals.

Escherichia coli is defined as the bacteria judged to have β-glucuronidase activity by a specified enzyme substrate medium method. E. coli is permanently present in the intestinal tract of a human or a warm-blooded animal, and is reliable as an indicator of fecal pollution compared to the coliforms not originating from feces. Further, E. coli is not only a single bacterial species but also has a short survival time in the natural environment compared to other fecal indicator bacteria, and accordingly can be used as a more specific indicator of fecal pollution.

Further, the fecal coliforms among coliforms are defined as bacteria which form blue colonies on the M-FC medium on incubation at 44.5° C. for 24 hours or produce a gas in the EC medium, and are mainly constituted of Escherichia genus and part of Citrobacter genus, Enterobacter genus and Klebsiella genus. In a fecal pollution-free environment, the fecal coliforms are hardly present even when coliforms are present. Thus, the fecal coliforms are less reliable as an indicator of fecal pollution than E. coli, but in most environments, the concentration of the fecal coliforms is closely related to the concentration of E. coli. Thus, the fecal coliforms are thought to be superior to coliforms as an indicator of fecal pollution.

The Ministry of Health, Labor and Welfare (Japan) employs E. coli as the fecal pollution indicator in the water quality standards for drinking water, and the Ministry of Land, Infrastructure and Transport (Japan) employs coliforms as the fecal pollution indicator in the effluent standards in a sewage treatment plant. Examination in accordance with each of the targets for measurement is obligatory and test methods are also stipulated. Thus, it is necessary to measure the number of E. coli or the number of the coliforms in a sample on the basis of the standards regulated in accordance with the types of test samples.

The test method stipulated in a field of the examination of E. coli and the coliforms in the sample is a method for counting the number of colonies after cultured in a liquid medium or on an agar medium. These methods, however, need more than 24 hours to judge the number of E. coli and the coliforms. Further, these methods require preparing a plurality of fermentation tubes or medium and have less working efficiency.

As a method of judging the number of the bacteria in a sample, various methods have been proposed in order to improve shortening of time and working efficiency. For example, a method of calculating the number of the bacteria in a sample by amplifying a specific gene for E. coli by polymerase chain reaction, and thereafter detecting them (PCR), a method of counting the number of the bacteria in a sample by measuring the amount of an enzyme labeled specific antibody for E. coli by fluorescent or chemiluminescence reaction (enzyme immunoassay) and the like are proposed. However, since these methods handle viable bacteria and dead bacteria at the same level, it is difficult to make a distinction between life and death, and accordingly the measurement result can hardly be used as a hygienic indicator. Further, there is proposed a method of simply incubating the bacteria in a sample in a liquid medium or on an agar medium to allow them to form micro colonies, and counting the formed micro colonies by utilizing chemiluminescence and fluorescence. However, according to this method, in order to raise the number of bacteria higher than the background in the measurement, a pre-incubation of four hours or more is required. Furthermore, there is provided a method of measuring the presence or absence of coliforms by placing a sample in a sealed container containing a medium for inhibiting the growth of bacteria other than coliforms such as a desoxycholate medium, and measuring the change of oxygen or carbon dioxide within the container based on the respiration of coliforms (respiratory activity method), but this method cannot rapidly detect the coliforms in a low concentration sample due to the lowering of the measurement sensitivity in inverse proportion to the number of bacteria.

In order to solve these problems, Fiksdal et al. proposed a method of rapidly counting the number of coliforms by measuring the activity of β-glucuronidase of an enzyme which E. coli inherently possesses as the method of measuring the number of the E. coli in a sample (Fiksadal, L., Pommepuy, M., Caprais, M.-P., and Midttun, I., “Monitoring of fecal pollution in coastal waters by use of rapid enzymatic technique”, Appl. Environ. Microbiol., 60, 1581-1584 (1994)). This method first subjects a sample of the target of measurement to optional concentration treatment, then performs lysis treatment to extract an enzyme (β-glucuronidase) from E. coli in the sample, subsequently adds an enzyme reaction substrate specific to this β-glucuronidase to cause fluorescence or the like and measures the fluorescence or the like to quantify the number of the coliforms in the sample. This method (enzyme activity method) proposed by Fiksdal et al. has been variously modified up to now (Van Poucke, S. O., and Nelis, H. J., “Rapid detection of fluorescent and chemiluminescent total coliforms and Esherichia coli on membrane filters”, J. Appl. Microbiol., 42, 233-244 (2000), and George, I., Crop, P., and Servais, P., “Use of β-D-galactosidase and β-D-glucuronidase activities for quantitative detection of total and fecal coliforms in wastewater”, Can., J. Microbiol., 47, 670-675 (2001)). Rompre el al. evaluated this enzyme activity method proposed by Fiksdal et al. as “a suitable substitute means for the conventional methods”, compared to the methods of rapidly measuring E. coli/coliforms such as the PCR method and the immunoassay (Rompre, A., Servais, P., Baudart, J., de-Roubin, M.-R., and Laurent, P., “Detection and enumeration of coliforms in drinking water: current methods and emerging approaches”, J. Microiol., Meth., 49, 31-54 (2002)).

The biggest problem for actually working the enzyme activity method proposed by Fiksdal et al. is an improvement of the detection sensitivity. The sewage discharge standard value of coliforms is 3×10³ CFU/mL. In general, the analytical method is required to have a measurement sensitivity of 1/10 of the standard value. Further, the number of E. coli in the sewage discharge water is about 1/10 of the number of coliforms. Thus, for the E. coli, the detection sensitivity of 3×10¹ CFU/mL is demanded. However, according to the method of measuring E. coli by the conventional enzyme activity method, satisfactory sensitivity could have not been obtained. The widely used enzyme reaction substrate for measuring glucuronidase activity of E. coli is 4-methylumbelliferyl-β-glucuronide (MUGLu) of a fluorescent reaction substrate. However, according to the technique using MUGLu (George, I., Crop, P., and Servais, P., “Use of β-D-galactosidase and β-D-glucuronidase activities for quantitative detection of total and fecal coliforms in wastewater”, Can., J. Microbiol., 47, 670-675 (2001)), the lower limit value of detection is 3×10³ CFU/mL and the socially demanded detection sensitivity has not been obtained. It is generally regarded that the luminescence measurement can obtain higher sensitivity than the fluorescence measurement. In order to improve the sensitivity, a novel luminescent enzyme reaction substrate directed to β-glucuronidase has been proposed (Japanese Patent Publication A No. 2000-270894). However, according to the measuring method proposed by Japanese Patent Publication A No. 2000-270894, the lower detection limit is as much as 2×10⁵ CFU/mL and is not suitable for practical purposes. Furthermore, in order to further improve the lower limit value of detection in the micro colony method, there is proposed a technique with the use of 3-(4-methoxyspiro-{1,2-dioxetan-3,2′-(5′-chloro)-tricyclo-[3.3.1.1^{3,7}]decan}-4-yl)phenyl-β-D-glucuronide (Glucuron: registered trademark, Tropix, Inc.) (Japanese Patent Publication A No. H11-505405). However, this technique requires increasing the number of bacteria (or forming micro colonies) by a pre-incubation of 6.5 hours to cause a problem of rapidness.

Further, in the detection/quantification of bacteria such as E. coli and coliforms, the improvement of the analytical sensitivity is tried by performing the concentration of a sample by passing a sample fluid containing bacteria through a filter membrane to collect the bacteria on the filter membrane and analyzing these collected (concentrated) bacteria. As the filter membrane which is used for this purpose, in general, membrane filters having a pore diameter of 0.2 to 0.45 μm have been conventionally used (Japanese Patent Publication A No. H11-505405 and Japanese Patent Publication A No. H05-273217). The reason is that the size of E. coli is generally thought to be about 1 to 2 μm×0.2 to 0.4 μm, and without using the filter membranes having a pore diameter in the above described range, E. coli could not be collected in high yield.

However, when the bacteria in sample water were collected by the filter membranes having a pore diameter in the above described range to perform analysis, the background value of the result of analysis was high and the lower limit value of detection was high. This is thought to be due to the reason that with the filter membranes having a pore diameter in the above described range, substances which are present in the sample and adversely affect the analysis, that is, foreign matters (background) which react with an enzyme reaction substrate to affect the measured enzyme activity, have been collected on the filter membranes in addition to the bacteria of the target for analysis. On this account, particularly for a sample containing low concentration E. coli, the detection/quantification of E. coli by this analytical technique was difficult.

### SUMMARY OF INVENTION

An object of the present invention is to solve the above described problems of the conventional art and to provide a method and an apparatus which can rapidly and simply detect and quantify bacteria, particularly Escherichia coli, coliforms and the like in a sample with high sensitivity.

As a result of strenuous investigations focusing on the relationship between the pore diameter of a filter membrane and the detecting performance in the technique of collecting bacteria such as E. coli and coliforms with a filter membrane to analyze (detect and quantify) them by the enzyme activity method, the present inventors have found that the use of a filter membrane having a specified pore diameter enables remarkable reduction in the analytical background value due to the presence of foreign matters and high sensitivity detection and quantification of bacteria, and were thus led to complete the present invention. Specifically, the present inventors have found that the collection of E. coli and the like is affected by not only the pore diameter of a filter membrane but also the adsorption of a filter membrane, and further that membranes having a pore diameter of 0.6 to 5.0 μm, preferably 0.8 to 3.0 μm and adsorptive property are most suitable for eliminating foreign matters to selectively collect E. coli alone, contrary to the common sense heretofore (that filter membranes having a pore diameter of about 0.2 to 0.45 μm are thought to be most suitable for collection of E. coli and the like), and completed the invention.

Thus, according to the present invention there is provided a method of detecting bacteria comprising passing a sample fluid through a filter membrane having a pore diameter of 0.6 to 5.0 μm and/or a filter membrane having a flow rate of distilled water passed, measured in accordance with ASTM F317-72, of 50 to 500 mL/min·cm² to collect the bacteria in the sample fluid on the filter membrane, bringing the bacteria collected filter membrane into contact with a lysing agent and an enzyme reaction substrate and finding an enzyme activity of the enzyme in the target bacteria to the enzyme reaction substrate to quantify the number of bacteria in the sample fluid. The enzyme activity can be found on the basis of the quantity of luminescence, the quantity of fluorescence, the quantity of color developing or the like, of a reaction product of the enzyme in the target bacteria with the enzyme reaction substrate. As to the contact of the filter membrane with the lysing agent and the enzyme reaction substrate, the filter membrane may be simultaneously brought into contact with the lysing agent and the enzyme reaction substrate or the filter membrane may be first brought into contact with the lysing agent, and thereafter brought into contact with the enzyme reaction substrate.

Furthermore, the present inventors have found that the analytical sensitivity can be further improved by passing a surfactant through the bacteria collected filter membrane to wash the bacteria collected filter membrane with a surfactant in collecting bacteria with the use of the above described specific filter membrane to detect/quantify them by the enzyme activity method.

In other words, another embodiment of the present invention relates to the above described method of detecting the bacteria in a sample fluid further comprising passing a surfactant through the bacteria collected filter membrane to wash the filter, and thereafter measuring the enzyme activity of the bacteria collected on the filter member.

Furthermore, the present inventors have found that with the use of 3-(4-methoxyspiro{1,2-dioxetan-3,2′-(5′-chloro)-tricyclo-[3.3.1.1^{3,7}]decan}-4-yl)phenyl-β-D-glucuronide or its derivative (for example, Glucuron: registered trademark or the like) as the enzyme reaction substrate, β-glucuronidase activity is measured and by calculating the number of E. coli and/or coliforms and/or fecal coliforms from the measured β-glucuronidase activity, the number of E. coli, coliforms and/or fecal coliforms can be detected with high sensitivity. The measuring time of β-glucuronidase activity by the method of the present invention is about 10 minutes to about one hour, and could remarkably shorten the measuring time (6.5 hours) in the conventional method as disclosed in Japanese Patent Publication A No. H11-505405. Further, the measuring sensitivity (lower limit value of detection) is as high as 0.1 CFU/mL to 2.5×10¹ CFU/mL in the number of E. coli and is remarkably improved from the lower limit value (1,000 CFU/mL) of the method disclosed in Japanese Patent Publication A No. H11-505405.

That is, another embodiment of the present invention relates to the above described method of detecting bacteria, particularly E. coli and the like in the sample fluid further comprising measuring β-glucuronidase activity with the use of 3-(4-methoxyspiro{1,2-dioxetan-3,2′-(5′-chloro)-tricyclo-[3.3.1.1^{3,7}]decan}-4-yl)phenyl-β-D-glucuronide or its derivative (for example, Glucuron: registered trademark or the like) as the enzyme reaction substrate.

Furthermore, the present inventors have found that by measuring β-galactosidase activity with the use of an enzyme reaction substrate specific to β-galactosidase, for example, 3-(4-methoxyspiro{1,2-dioxetan-3,2′-tricyclo-[3.3.1.1^{3,7}]decan}-4-yl)phenyl-β-D-galactopyranoside or its derivative, particularly the chloro derivative (Galacton: registered trademark; Tropix, Inc. or the like) of a chemiluminogenetic enzyme reaction substrate and calculating the number of E. coli in a sample from the measured β-galactosidase activity, the number of coliforms can be detected with high sensitivity.

In the method of the present invention, first, a water sample of the target for measurement is passed through a membrane filter having a pore diameter of 0.6 to 5.0 μm and/or a membrane filter having a flow rate of distilled water passed, measured in accordance with ASTM F317-72, of 50 to 500 mL/min·cm² to collect the bacteria, particularly E. coli, coliforms and/or fecal coliforms from the sample fluid on the filter membrane. Then, if desired, the membrane collected bacteria, particularly E. coli, coliforms and/or fecal coliforms are washed with a surfactant. Subsequently, this filter membrane is brought into contact with a lysing agent and an enzyme reaction substrate specific to the bacteria of the target for measurement (for example, β-glucuronidase in the case of E. coli and β-galactosidase in the case of coliforms or the like) or the lysing agent is previously added to this membrane, and thereafter the membrane is brought into contact with the enzyme reaction substrate. By this contact, enzyme substrate reaction occurs to produce a compound having a property such as luminescence, fluorescence and color developing capable of being quantitatively measured. Also, by adding an accelerator for accelerating luminescence or the like thereto to measure the quantity of luminescence or the like, the enzyme activity of the sample is measured. On the other hand, in the test target system, the correlation between the number of bacteria (E. coli, coliforms and/or fecal coliforms and the like) of the target for measurement in the sample and the enzyme activity is previously found to prepare a working curve. Further, on the basis of the correlation (the working curve) between the previously found number of bacteria and the enzyme activity, the number of the bacteria in the sample is calculated from the measured value of the measured enzyme activity.

In the method of the present invention, a water sample as such may be used but it is desirable to previously remove large particles by coarse filtration of the water sample with filter paper No. 5A (corresponding to an effective pore diameter of 7 μm) or a filter corresponding to this filter paper. In passing the sample through a filter, it is desirable to previously disperse the sample with the use of a means such as ultrasonic wave, and the recovery ratio is desirably increased by an operation such as suction/air-washing after passing the sample through the filter.

The filtrate (water sample), if necessary, obtained by performing coarse filtration is passed through a membrane filter having a pore diameter of 0.6 to 5.0 μm to collect the bacteria present in the sample fluid on the filter membrane. According to the investigations by the present inventors, it has been found that the use of a filter membrane having a pore diameter (average pore diameter) of 0.6 to 5.0 μm which has not been used heretofore in collecting bacteria such as E. coli enables high yield collection of E. coli and the like. In the present invention, a filter membrane having a pore diameter (average pore diameter) of 0.8 to 3.0 μm is preferably used for this purpose. Further, the pore diameter (average pore diameter) of a membrane filter is shown by the manufactures as a declared value in the tolerance range of about ±30%. As the material of membrane filters, a membrane filter made of nitrocellulose (a mixture of cellulose nitrate and cellulose acetate: cellulose mixed ester), a membrane filter made of cellulose acetate, a ceramic filter, a metal mesh filter, a fabric filter and the like can be used, and a nitrocellulose membrane filter excellent in adsorbability is most preferred.

It is known that there is a correlation between the pore diameter of a filter membrane and the flow rate of distilled water passed, and the measuring method is regulated. According to the present invention, it has been found that membranes having a flow rate of distilled water passed, measured by the testing method used in the product standards of a membrane which is ASTM F317-72 “Standard Test Method for Liquid Flow Rate of Membrane Filter” (distilled water of 25° C. from which particles have been removed by filtering with a membrane filter having a pore diameter of 0.10 μm is filtered under a reduced pressure of −0.069 MPa and its flow rate is expressed by the number of mL per minute per 1 cm²), of 50 to 500 ml/min·cm² are suitable.

As a result of the investigations by the present inventors, it has been found that as the filter membrane which can be used in collecting E. coli and the like in high yield according to the present invention, the filter membranes having a flow rate of distilled water passed, in the liquid flow test of ASTM F317-72 using the above described method, of 50 to 500 ml/min·cm², preferably 120 to 300 ml/min·cm² , are suitable.

Further, in the case of using membrane filters having a large pore diameter (a flow rate of water passed of more than 200 mL/cm²), by using a plurality of filter membranes in a superposed relation with one another, the collection efficiency of bacteria can be improved and such an embodiment is included in the scope of the present invention.

Further, in the case of using membranes having a small thickness (a thickness of 50 μm or less, particularly a thickness of 10 μm or less), the recovery ratio of bacteria is lowered, and accordingly membranes having a rather small pore diameter are preferably used (membranes having a rather large pore diameter (a flow rate of water passed of more than 150 mL/cm² are preferably not used).

Then, the filter membrane is brought into contact with a lysing agent and an enzyme reaction substrate to measure the activity of the enzyme extracted from the bacteria (E. coli and/or coliforms) collected on the filter membrane. As to the contact of the filter membrane with the lysing agent and the enzyme reaction substrate, the filter membrane may be brought into contact with the lysing agent and the enzyme reaction substrate together, or first, the filter membrane may be brought into contact with the lysing agent to extract an enzyme, and thereafter the extracted enzyme may be brought into contact with the enzyme reaction substrate.

In the present invention, before the membrane filter is brought into contact with the lysing agent and the enzyme reaction substrate, the filter membrane is preferably washed with a surfactant having a concentration of not lysing bacterial bodies. From the investigations by the present inventors it has been found that, by washing the bacteria collected filter membrane with a surfactant, the background in the analysis by the enzyme activity method can be further reduced to remarkably improve the analytical sensitivity. The surfactant to be used for this purpose includes, for example, a nonionic surfactant such as Triton X-100 (trade name) and an anionic surfactant such as sodium dodecyl sulfate (SDS), and these may be preferably used alone or combined thereof. The concentration of the surfactant, for example, in the case of Triton X-100, is preferably 0.01 to 0.1 vol. %.

By bringing the filter membrane into contact with the lysing agent and the enzyme reaction substrate, the enzyme having the correlation with the number of the target bacteria is extracted to cause a specific enzyme reaction, and the enzyme activity is measured as the indicator of the enzyme reaction product, and thus the bacteria can be detected/quantified. The enzyme in a correlation with the number of bacteria which can be used is, for example, β-glucuronidase in the case of E. coli of the target bacteria and β-galactosidase and/or β-glucuronidase in the case of coliforms and/or fecal coliforms of the target bacteria.

As the lysing agent, a nonionic surfactant such as Triton X-100 having a concentration capable of lysing bacterial cells and an anionic surfactant such as sodium dodecyl sulfate (SDS) are preferably used alone or combined. The concentration of the surfactant capable of lysing bacterial cells is, for example, preferably in the range of 0.05 to 0.5 vol. % in the case of E. coli and coliforms. When lysis is performed by adding a surfactant as the lysing agent, room temperature may be adequate, but a constant temperature between 20 to 45° C. is preferable for performing lysis. As described above, when bacteria are collected on a filter membrane and then enzyme activity test for the collected bacteria is carried out, the lysis treatment with a surfactant is preferably performed in the state of the bacteria collected filter membrane dipped in the surfactant while maintaining this state for 5 to 60 minutes, preferably for 10 to 30 minutes. The enzyme reaction substrate may be added together with the lysing agent or may be separately added after the lysis treatment.

Further, before adding the lysing agent and the enzyme reaction substrate, ultrasonic crushing can be also performed. In the case of performing the lysis treatment by ultrasonic crushing, when, after a surfactant is further added to the ultrasonic treated liquid, and the enzyme reaction substrate is added thereto to allow the enzyme substrate reaction to proceed, it is preferred that the bacterial cells are solubilized to advance the dispersion of the extracted enzyme, and accordingly the sensitivity is improved. In this case, the surfactant is added to the ultrasonic treated fluid in such an amount as to render its concentration 0.01 to 0.1 vol. % which can disperse the enzyme and, simultaneously, does not inhibit the enzyme reaction.

The enzyme substrate reaction (the reaction of an enzyme with an enzyme reaction substrate) is preferably carried out in a buffer solution. As the buffer solution to be used for this purpose, a phosphate buffer solution having a pH adjusted to 6.0 to 8.5, PBS (phosphate buffered saline) and the like can be used and, if necessary, the buffer solution added with 10 to 200 mM EDTA and 0.02 to 2 wt. % BSA (bovine serum albumin) can be also used. It is preferred that the enzyme reaction substrate is mixed with the extracted enzyme sample and allowed to react therewith at a constant temperature for 5 to 60 minutes. The reaction temperature may be any temperature in the range of 20° C. to 45° C. and preferably 30° C. to 40° C.

As the enzyme reaction substrate, for example, when the enzyme having a correlation with the number of bacteria of the target is β-glucuronidase, 4-methylumbelliferyl-β-D-glucuronide, 5-bromo-4-chloro-3-indolyl-β-D-glucuronide and 4-trifluoroumbelliferyl-β-D-glucuronide as fluorogenic enzyme reaction substrates; 3-(4-methoxyspiro{1,2-dioxetan-3,2′-(5′-chloro)-tricyclo-[3.3.1.1^{3,7}]decan}-4-yl)phenyl-β-D-glucuronide [Glucuron (registered trademark)] and D-luciferin-O-β-D-glucuronide as chemiluminogenetic enzyme reaction substrates; p-nitrophenylglucuronide as a color developing enzyme reaction substrate; and the like can be used. Further, when the enzyme having a correlation with the number of bacteria of the target is β-galactosidase, 4-methylumbelliferyl-β-D-galactopyranoside and 4-trifluoromethylumbelliferyl-β-D-galactopyranoside as fluorogenetic enzyme reaction substrates; 3-(4-methoxyspiro{1,2-dioxetan-3,2′-tricyclo-[3.3.1.1^{3,7}]decan}-4-yl)-phenyl-β-D-galactopyranoside and its derivative, particularly the chloro derivative (Galacton: registered trademark, Tropix, Inc.) and D-luciferin-O-β-D-galactoside as chemiluminogenetic enzyme reaction substrates; and the like can be used.

The filter membrane is dipped in a buffer solution containing an enzyme reaction substrate (also referred to as “an enzyme reaction substrate fluid”) and is allowed to react at a constant temperature for 5 to 60 minutes and in this instance, the amount of the enzyme reaction substrate fluid added is preferably 0.1 to 5 mL. In this case, it is preferred that by using a reactor vessel having a bottom area slightly larger than the area of the filter membrane and horizontally placing the filter membrane in the vessel, the filter membrane can be dipped in a smaller amount of the enzyme reaction substrate liquid. At this time, the reaction temperature may be any temperature between 20° C. and 45° C. and preferably between 30° C. and 40° C., and appropriate stirring is preferably applied.

The reaction of an enzyme with an enzyme reaction substrate produces a luminescent, fluorescent or color developing compound. After the reaction, if necessary, an accelerator (a light emission accelerator, a fluorescence accelerator or a color developing accelerator) is added to cause luminescence or the like, and its quantity is measured. In the case of measuring the quantity of chemiluminescence, the mixing time with the accelerator is preferably set at 10 seconds or less, more desirably one second or less. It is preferred that the waiting period for measuring the quantity of chemiluminescence or the like from the addition of the accelerator is set at one second to one minute, more desirably two seconds to 10 seconds. The mixing time, waiting period and measuring time are always rendered constant among samples.

As to the measurement of the quantity of chemiluminescence, part of the reaction fluid or the entire reaction fluid after the enzyme reaction is mixed with a light emission accelerator to measure the quantity of chemiluminescence. In this instance, part of the reaction fluid after the enzyme reaction is collected and the light emission accelerator is added thereto, and then the intensity of luminescence can be measured but it is preferred that the light emission accelerator is directly added to the filter membrane dipped in the enzyme reaction substrate fluid, and the quantity of luminescence is measured from the filter membrane. At this time, the direction of photometry is not limited, and the photometry from the direction facing the filter membrane is preferred. In the method utilizing fluorescence and calorimetric intensity, it is necessary to irradiate with excitation light and transmitted light, and a solid substance such as a filter membrane which disturbs the transmission of light is not allowed to be present in the reaction fluid but in the case of the measurement of chemiluminescence, it is unnecessary to irradiate with excitation light and furthermore, due to the reflection of the generated light from the filter membrane, more efficient and stable measurement is possible. Further, the filter membrane is preferably white in color, but when nonspecific luminescence (background) is recognized, a black filter membrane is preferably used.

Further, the present inventors have found that in the enzyme activity method of measuring the number of E. coli based on the correlation between the β-glucuronidase activity measured value and the number of E. coli in a test sample, the number of E. coli can be quantified with high sensitivity by using 3-(4-methoxyspiro{1,2-dioxetan-3,2′-(5′-chloro)-tricyclo-[3.3.1.1^{3,7}]decan}-4-yl)-phenyl-β-D-glucuronide or its derivative, for example, Glucuron (registered trademark) and the like as the enzyme reaction substrates for measuring the β-glucuronidase activity, allowing this enzyme reaction substrate to react with β-glucuronidase to produce a luminescence, allowing the produced luminescence to emit light with the use of an accelerator containing a cationic polymer and an alkali, quantifying the quantity of luminescence to measure the β-glucuronidase activity and calculating the number of E. coli in the sample from the measured β-glucuronidase activity. Further, E. coli is present in coliforms and fecal coliforms, and the existing ratio of E. coli in coliforms and fecal coliforms is almost constant depending on the types of samples, and thus, by measuring the concentration of E. coli, the number of coliforms and fecal coliforms can be estimated.

Further, the present inventors have found that in the enzyme activity method of estimating the number of coliforms and/or fecal coliforms based on the correlation between the β-galactosidase activity measured value and the number of coliforms and/or the number of fecal coliforms in a test sample, 3-(4-methoxyspiro{1,2-dioxetan-3,2′-(5′-chloro)-tricyclo-[3.3.1.1^{3,7}]decan}-4-yl)phenyl-β-D-galactopyranoside or its derivative, for example, Galacton (registered trademark) and the like are used as the enzyme reaction substrates for measuring β-galactosidase and allowed to react with β-galactosidase to produce a luminescence, and the produced luminescence is allowed to emit light with the use of a light emission accelerator containing a cationic polymer and an alkali, for example, Accelerator II (trade name), and the quantity of luminescence is quantified to measure β-galactosidase activity, and from the measured β-galactosidase activity, the number of coliforms and/or the number of fecal coliforms can be estimated with high sensitivity.

The above described embodiment of measuring the β-glucuronidase activity with the use of 3-(4-methoxyspiro{1,2-dioxetan-3,2′-(5′-chloro)-tricyclo-[3.3.1.1^{3,7}]decan}-4-yl)phenyl-β-D-glucuronide or its derivative, for example, Glucuron (registered trademark) as the enzyme reaction substrates and the above described embodiment of measuring the β-galactosidase activity with the use of 3-(4-methoxyspiro{1,2-dioxetan-3,2′-(5′-chloro)-tricyclo-[3.3.1.1^{3,7}]decan}-4-yl)phenyl-β-D-galactopyranoside or its derivative, for example, Galacton (registered trademark) as the enzyme reaction substrates are included in the scope of the present invention. Specifically, a more preferred embodiment of the present invention relates to a method for detecting bacteria in a sample by the enzyme activity method above described wherein the target bacteria for measurement is E. coli and 3-(4-methoxyspiro{1,2-dioxetan-3,2′-(5′-chloro)-tricyclo-[3.3.1.1^{3,7}]decan}-4-yl)phenyl-β-D-glucuronide or its derivative is used as enzyme reaction substrates to the β-glucuronidase to be extracted from E. coli, or wherein the target bacterium for measurement are coliforms and/or fecal coliforms and 3-(4-methoxyspiro{1,2-dioxetan-3,2′-(5′-chloro)-tricyclo-[3.3.1.1^{3,7}]decan}-4-yl)phenyl-β-D-galactopyranoside or its derivative, for example, Galacton (registered trademark) is used as enzyme reaction substrates to β-galactosidase to be extracted from E. coli and/or coliforms.

In the embodiment of measuring β-glucuronidase activity with the use of 3-(4-methoxyspiro{1,2-dioxetan-3,2′-(5′-chloro)-tricyclo-[3.3.1.1^{3,7} ]decan}-4-yl)phenyl-β-D-glucuronide or its derivative as enzyme reaction substrates or in the embodiment of measuring β-galactosidase activity with the use of 3-(4-methoxyspiro{1,2-dioxetan-3,2′-(5′-chloro)-tricyclo-[3.3.1.1^{3,7}]decan}-4-yl)phenyl-β-D-galactopyranoside or its derivative, for example, Galacton (registered trademark), it is preferred that after the enzyme substrate reaction, the addition of a light emission accelerator causes chemiluminescence to measure the quantity of luminescence. As the light emission accelerator which can be used in such embodiments, an agent comprising a cationic polymer and an alkali of a poly(vinylbenzyl-benzyldimethyl)ammonium chloride-containing alkali solution can be used and, for example, Accelerator II (trade name), commercially available from Tropix, Inc. can be used. Further, in the case of using a chloro derivative of 3-(4-methoxyspiro{1,2-dioxetan-3,2′-(5′-chloro)-tricyclo-[3.3.1.1^{3,7}]decan}-4-yl)-phenyl-β-D-galactopyranoside, for example, Galacton-Star (registered trademark; Tropix, Inc.) and the like as enzyme reaction substrate for β-galactosidase, the quantity of luminescence can be measured without using the light emission accelerator.

As the method of calculating the enzyme activity from the quantity of luminescence, the quantity of fluorescence, the quantity of color development and the like, any suitable technique known in the art can be employed. Further, generally, the enzyme activity is expressed by the unit of the amount of the produced and increased reaction product per unit time. However, in the case of using 3-(4-methoxyspiro{1,2-dioxetan-3,2′-(5′-chloro)-tricyclo-[3.3.1.1^{3,7}]decan}-4-yl)phenyl-β-D-glucuronide or its derivative, for example, Glucuron (registered trademark) as the enzyme reaction substrate, it is difficult to express the enzyme activity by the amount of the product per unit time due to the difficulty in directly quantifying the reaction product. Then, in the present invention the amount of the reaction product is replaced with the quantity of light emission by a luminometer and the enzyme activity is expressed by the quantity of increasing light emission per unit time. Specifically, the enzyme activity is expressed by RLU/min (Relative Light Unit, relative quantity of light emission increased per minute). In the following, the quantity of light emission increased per unit time (RLU/min) is taken as the enzyme activity. Further, it is also possible to take the intensity of luminescence after a specified time of the reaction under the same measuring conditions as the enzyme activity.

A working curve can be prepared by plotting the relationship between the measured value of the β-glucuronidase activity found by the same enzyme activity measuring method obtained beforehand with the use of various predetermined concentrations of bacterial samples in the same system of the test sample of the target for measurement, and the number of colonies obtained by incubating the bacteria in the samples in a liquid medium or on an agar medium and counting the number of colonies. Also, based on this working curve, the number of bacteria of the target for measurement can be quantified from the measured value of the enzyme activity relating to an unknown sample by the above described enzyme activity measuring method.

As described above, according to the present invention, a sample fluid is passed through a filter membrane having a pore diameter of 0.6 to 5.0 μm and/or a flow rate of distilled water of 50 to 500 mL/min·cm² to collect bacteria in the sample fluid on the filter membrane, and the bacteria collected filter membrane is brought into contact with a lysing agent and an enzyme reaction substrate to measure an enzyme activity by the enzyme reaction of the bacteria, and thus the analytical background value due to the presence of foreign matters can be remarkably reduced and the bacteria in the sample, for example, E. coli and coliforms can be rapidly and simply detected/quantified with high sensitivity.

Further, the present invention relates to an apparatus for carrying out the above explained method of detecting the bacteria in a sample.

According to the present invention, there is provided an apparatus for detecting bacteria (preferably E. coli and/or coliforms) comprising: a collection vessel comprising a filter membrane having a pore diameter of 0.6 to 5.0 μm and/or a flow rate of distilled water passed, measured in accordance with ASTM F317-72, of 50 to 500 mL/min·cm² ; a sample fluid feed mechanism for feeding a sample fluid to the primary side of the filter membrane; an enzyme reaction substrate feed mechanism for feeding an enzyme reaction substrate; and an enzyme activity measuring device for measuring an enzyme activity of an enzyme in the target bacteria to the enzyme reaction substrate. The apparatus for measuring bacteria of the present invention may further comprise a washing agent feed mechanism, a lysing agent feed mechanism and/or an accelerator feed mechanism. The sample fluid feed mechanism and enzyme reaction substrate feed mechanism, and washing agent feed mechanism, lysing agent feed mechanism and/or accelerator feed mechanism are preferably connected to the collection vessel by piping having a valve and the like so as to feed, preferably quantitatively feed each fluid, if necessary. Further, the apparatus for detecting bacteria of the present invention may comprise a reaction vessel for advancing the enzyme reaction of the collection vessel separately. Alternatively, the enzyme reaction may be advanced in the collection vessel. Since, in order to advance the enzyme reaction, it is necessary to maintain the enzyme reaction at 30° C. to 40° C., the collection vessel and/or reaction vessel is preferably installed in a thermostatic chamber.

Further, according to the present invention, there is provided an apparatus for detecting the bacteria in a sample fluid comprising a collection unit for collecting the bacteria from the sample fluid, a reaction unit for allowing enzyme substrate reaction to proceed and a means for transferring an enzyme extracted fluid from the collection unit to the reaction unit; wherein the collection unit comprises: a filter device including a filter membrane having a pore diameter of 0.6 to 5.0 μm and/or a filter membrane having a flow rate of distilled water passed, measured in accordance with ASTM F317-72, of 50 to 500 mL/min·cm² ; a sample feed device for feeding the sample fluid to a primary side of the filter membrane; a washing solution feed device for feeding washing solution to the primary side of the filter membrane; and a means for extracting an enzyme from the bacteria collected on the filter membrane; and the reaction unit comprises a reaction vessel for receiving the enzyme extracted fluid to allow enzyme substrate reaction; an enzyme reaction substrate adding device for feeding an enzyme reaction substrate to the enzyme extracted fluid in the reaction vessel; an accelerator adding device for adding an accelerator to the enzyme reaction substrate added reaction fluid in the reaction vessel; and a measuring device for measuring the quantity of luminescence, fluorescence or color developing of the accelerator added reaction liquid.

Still further, according to the present invention, there is provided an apparatus for detecting bacteria (preferably E. coli and/or coliforms) comprising a collection/reaction unit, a measuring unit and a means for transferring a reaction fluid after enzyme reaction from the collection/reaction unit to the measuring unit. The collection/reaction unit comprises a collection and reaction vessel for housing a filter membrane having a pore diameter of 0.6 to 5.0 μm and/or a filter membrane having a flow rate of distilled water passed, measured in accordance with ASTM F317-72, of 50 to 500 mL/min·cm², a sample feed device for feeding a sample fluid to a primary side of the filter membrane, a washing solution feed device for feeding a washing solution to the primary side of the filter membrane, a lysis enzyme reaction substrate adding device for feeding a lysis enzyme reaction substrate to the primary side of the filter membrane and a thermostatic device for maintaining the lysis enzyme reaction substrate fluid at a constant temperature in the state of the filter membrane dipped in the lysis enzyme reaction substrate fluid. The measuring unit comprises a measuring vessel for receiving the reaction fluid after enzyme reaction, an accelerator adding device for adding an accelerator to the reaction fluid after the enzyme reaction in the measuring vessel and a measuring device for measuring the quantity of luminescence, fluorescence or color developing of the accelerator added reaction fluid.

Furthermore, according to the present invention there is provided a kit for simplified measurement of bacteria (preferably E. coli and/or coliforms) comprising a collection/reaction vessel equipped with a feed inlet and a discharge outlet comprising a filter membrane having a pore diameter of 0.6 to 5.0 μm and/or a filter membrane having a flow rate of distilled water passed, measured in accordance with ASTM F317-72, of 50 to 500 mL/min·cm², an enzyme reaction substrate fluid feed unit and an enzyme activity measuring unit for measuring an enzyme activity. This kit may further comprise a washing agent feed unit, a lysing agent feed unit and/or an accelerator feed unit.

### BRIEF DESCRIPTION OF DRAWINGS

FIG. 1 is a diagram showing an example constitution of the apparatus for carrying out the method of the present invention.

FIG. 2 is a diagram showing another embodiment of the apparatus for carrying out the method of the present invention.

FIG. 3 is a diagram showing another embodiment of the apparatus for carrying out the method of the present invention.

FIG. 4 is a diagram showing another embodiment of the apparatus for carrying out the method of the present invention.

FIG. 5 is a diagram showing another embodiment of the apparatus for carrying out the method of the present invention.

FIG. 6 is a diagram showing another embodiment of the apparatus for carrying out the method of the present invention.

FIG. 7 is a diagram showing another embodiment of the apparatus for carrying out the method of the present invention.

FIG. 8 is a diagram showing another embodiment of the apparatus for carrying out the method of the present invention.

FIG. 9 shows one specific example of the collection device.

FIG. 10 is a diagram showing another embodiment of the apparatus for carrying out the method of the present invention.

FIG. 11 shows a specific example of the kit according to the present invention.

FIG. 12 are graphs showing the relationship between the pore diameter of each membrane filter and the efficiency of collecting bacteria measured in Example 1.

FIG. 13 are graphs showing the relationship between the pore diameter of each membrane filter and the relative background of the enzyme activity measured in Example 1. The enzyme activity (background) measured by performing the same enzyme activity test with a sample before filtration is taken as 100% and a relative value to this background is shown as the relative background.

FIG. 14 is one graph of the relative light unit (RLU) versus the enzyme substrate reaction time measured in Example 2. The reaction time is plotted on the abscissa and the relative light unit is plotted on the ordinate and the activity value is found from the gradient of a straight line.

FIG. 15 is a graph showing the relationship between the number of E. coli in the test sample (colony count value) and the β-glucuronidase activity measured in Example 2 according to the method of the present invention.

FIG. 16 is a graph showing the improvement of the detection limit value by membrane collection.

FIG. 17 is a graph showing the optimum conditions for washing substances collected by a filter membrane with a surfactant measured in Example 3 according to the method of the present invention.

FIG. 18 is a graph showing the relationship between the amount of the surfactant added in the enzyme activity test having performed lysis treatment by ultrasonic wave and the enzyme activity sensitivity measured in Example 4.

FIG. 19 is a graph showing the relationship between the lysing time in the enzyme activity test having performed lysis treatment with a surfactant and the enzyme activity sensitivity measured in Example 5.

FIG. 20 is a graph showing the relationship between the amount of the surfactant added in the enzyme activity test having performed lysis treatment with a surfactant and the enzyme activity sensitivity measured in Example 6.

FIG. 21 is a graph showing the relationship between the amount of the surfactant added and the enzyme activity sensitivity in the case of adding an enzyme reaction substrate and a lysing agent together measured in Example 7.

FIG. 22 shows one example of the graph of relative light unit (RLU) versus shaking time (enzyme substrate reaction) measured in Example 8.

FIG. 23 is a graph showing the relationship between the number of E. coli (colony count value) in the test sample and the β-glucuronidase activity measured in Example 8 according to the method of the present invention.

FIG. 24 is a graph showing the relationship between quantity of luminescence after specified reaction time and the concentration of E. coli.

FIG. 25 is a graph showing the relationship between the quantity of luminescence by direct photometry and preparative photometry and the number of E. coli.

FIG. 26 is a graph showing the relationship between the number of E. coli in the test sample and the β-glucuronidase activity measured in Example 10.

FIG. 27 is a graph showing the relationship between the number of coliforms in the test sample and the β-glucuronidase activity measured in Example 11 according to the method of the present invention.

FIG. 28 is a graph showing the relationship between the number of E. coli in the test sample and the β-glucuronidase activity measured in Example 12 according to the method of the present invention.

FIG. 29 is a graph showing the relationship between the number of fecal coliforms in the test sample and the β-glucuronidase activity measured in Example 13 according to the method of the present invention.

### DETAILED DESCRIPTION OF THE INVENTION

Preferred embodiments of the present invention will now be explained.

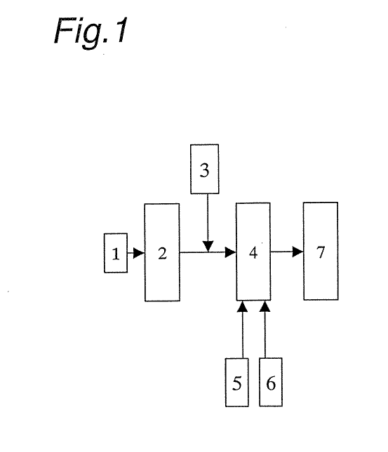

One embodiment of the present invention is an apparatus for detecting the bacteria in a sample fluid comprising: a collection vessel or filter which houses a filter membrane having a pore diameter of 0.6 to 5.0 μm and/or a flow rate of distilled water passed under the above described conditions of 50 to 500 mL/min·cm² and is connected to a sample fluid feed pipe on the primary side of the filter membrane; a lysis device for performing lysis treatment for the collected substances on the filter membrane of the filter; an enzyme substrate reactor having a means to receive the enzyme extract fluid to be obtained in the lysis treatment and to add an enzyme reaction substrate to a specific enzyme to allow enzyme substrate reaction to proceed; an accelerator adding device for adding an accelerator to the enzyme reaction added reaction fluid; and a measuring device for measuring the quantity of luminescence, fluorescence or color developing of the accelerator added reaction fluid. The concept of such an apparatus relating to this embodiment is shown in FIG. 1. A sample fluid 1 is first received in a filter 2. The filter 2 has an internal filter membrane, and the sample fluid 1 is fed to the primary side of the filter membrane and is passed therethrough and filtered. Next, lysis treatment is provided with the substances collected on the filter membrane by a lysis device 3. Specific examples of the lysis device 3 which can be employed include, for example, an ultrasonic irradiation device for irradiating the collected substances on the filter membrane with ultrasonic wave and a device for dipping the filter membrane which filters the sample fluid to extract an enzyme. Thereafter, the lysis treatment provided sample is added with an enzyme reaction substrate 5 to a specific enzyme in an enzyme substrate reactor 4, and then is added with an accelerator for accelerating luminescence, fluorescence or color developing of a luminescent, fluorescent or color developing compound produced by the enzyme substrate reaction from an accelerator adding device 6. Thus, luminescence or the like is caused, and the quantity of luminescence or the like is measured by a measuring device 7. On the basis of the quantity of luminescence measured in this manner, specific bacteria in the sample can be detected/quantified by the above described technique.

The filter 2 preferably further comprises a filter membrane washing solution feed device for feeding a surfactant washing solution to the primary side of the filter membrane in the filter which has filtered the sample fluid. Furthermore, the primary side of the filter membrane is preferably equipped with piping for cleaning the filter membrane with air.

Further, as a more specific embodiment of the apparatus for carrying out the present invention, another embodiment of the present invention relates to an apparatus for detecting the bacteria in a sample fluid comprising a collection unit for collecting the bacteria from the sample fluid, a reaction unit to allow enzyme substrate reaction to proceed and a means for transferring an enzyme extracted fluid from the collection unit to the reaction unit. The collection unit comprises a filter membrane having a pore diameter of 0.6 to 5.0 μm and/or a flow rate of distilled water passed under the above described conditions of 50 to 500 mL/min·cm², a sample feed device for feeding a sample fluid to a primary side of the filter membrane, a washing solution feed device for feeding a washing solution to the primary side of the filter membrane, and a means to extract an enzyme in the bacteria from the bacteria collected filter membrane. The reaction unit comprises a reaction vessel for receiving the enzyme extracted fluid to allow enzyme substrate reaction, an enzyme reaction substrate adding device for feeding an enzyme reaction substrate to the enzyme extracted fluid in the reaction vessel, an acceleration adding device for adding an accelerator to the enzyme reaction substrate fed reaction fluid in the reaction vessel and a measuring device for measuring the quantity of luminescence, fluorescence or color developing of the accelerator added reaction fluid.

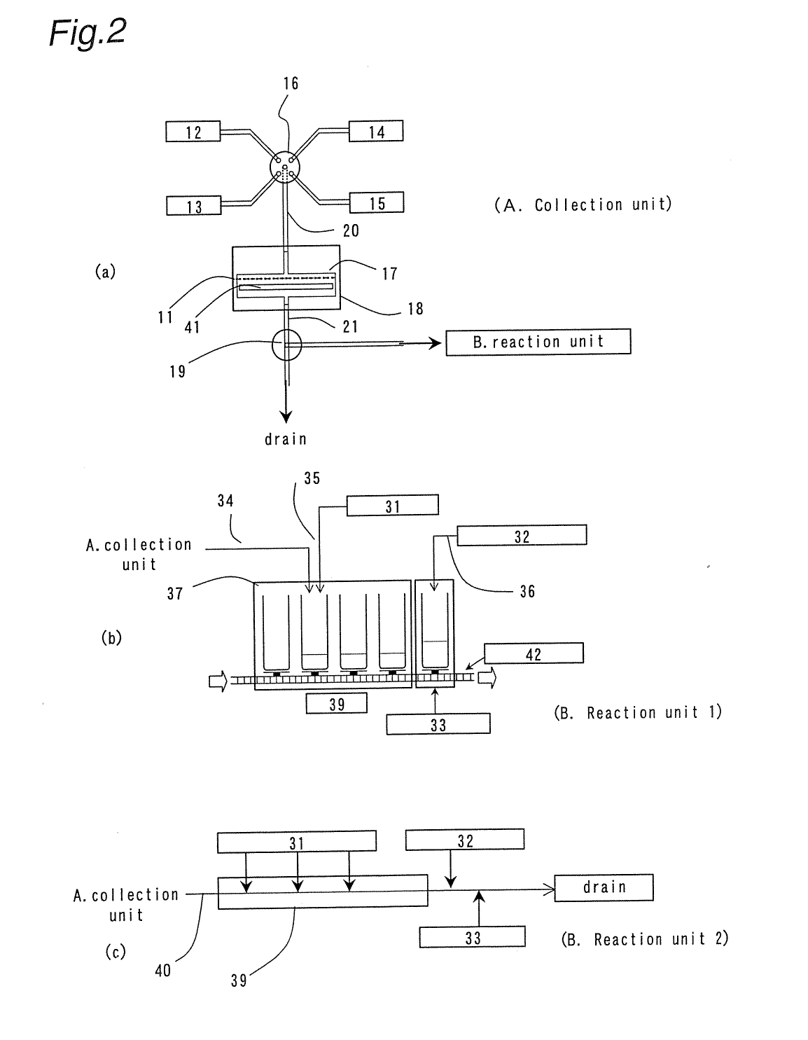

One specific example of the apparatus relating to such an embodiment is shown in FIG. 2. The apparatus for detecting the bacteria in a sample shown in FIG. 2 comprises a collection unit (A) for collecting the bacteria in the sample to perform lysis treatment and a reaction unit (B) for subjecting the lysis treatment provided sample in the collection unit (A) to the enzyme substrate reaction to quantitatively measure the quantity of luminescence or the like. In FIG. 2, two types of reaction units (B) are shown. In the reaction unit 1, reaction is allowed to proceed for every test sample, and accordingly the reaction unit 1 is suitable when the number of test samples is small. Further, in the reaction unit 2, the reaction is allowed to continuously proceed, and accordingly, the reaction unit 2 is suited for continuous measurement. It is possible to select the reaction unit in accordance with the object of measurement.

In FIG. 2, a filter 17 in the collection unit A houses a filter membrane 11 having a pore diameter of 0.6 to 5.0 μm and/or a flow rate of distilled water under the above described conditions of 50 to 500 mL/min·cm² and a perforated plate 41 for rendering the flow rate of water passed of the filter membrane constant. Feed piping 20 is connected to the primary side of the filter membrane 11 and is connected to an air feed device 12, a sample feed device 13, a lysing agent feed device 14 and a washing solution feed device 15 through a change-over valve 16. Further, a perforated plate 41 is connected to the back surface of the filter membrane 11 and a discharge pipe 21 is connected to the secondary side of the perforated plate 41. The filter 17 is arranged in a thermostatic chamber 18 and is preferably maintained, for example, at 37° C.

First, a sample fluid of the target for measurement from the sample feed device 13 is fed to the primary side of the filter membrane 11 at a constant flow rate and filtered by passing through the filter membrane. By this filtration, the bacteria from the sample fluid are collected on the filter membrane 11. The fluid passed through the filter membrane 11 is discharged as a drain via a valve 19 through a discharge pipe 21. Next, the change-over valve 16 is operated to change and a washing solution can be fed from the washing solution feed device 15 to the primary side of the filter membrane 11 to perform washing by passing water. The washing solution is preferably, for example, a nonionic surfactant such as Triton X-100. Washing solution passed through the filter membrane 11 is discharged as a drain via the valve 19 through the discharge pipe 21.

Then, the change-over valve 16 is operated to change and air can be blown from the air feed device 12. This air blowing purges the collection unit, and the washing solution remaining on the filter membrane can be discharged from the discharge pipe.

Next, the change-over valve 16 is operated to change and a lysing agent (a lysing fluid) is fed to the filter 17 from a lysing agent feed device 14. In this instance, the change-over valve 19 is closed and the lysing fluid is held within the filter 17 with the filter membrane 11 in a wet state with the lysing fluid for a specified time. Then, the change-over valve 19 is operated to change to connect the discharge pipe 21 to the reaction unit B and the change-over valve 16 is operated to change and air is blown from the air feed device 12 to allow the lysis treatment provided sample fluid to flow into the reaction unit B.

The embodiment of the reaction unit 1 shown in FIG. 2 (b) comprises a reaction vessel 37 for receiving the lysis treatment provided sample fluid from the collection unit A, an enzyme reaction substrate adding device 31 for adding an enzyme reaction substrate to the reaction vessel, an accelerator adding device 32 for adding an accelerator to the reaction vessel and a measuring device 33 for measuring the quantity of luminescence or the like of a luminescent, fluorescent or color developing compound to be produced by enzyme substrate reaction. The reaction vessel 37 is arranged within a thermostatic chamber 39 and is preferably maintained, for example, at 37° C.

The lysis treatment sample fluid from the collection unit A is fed to the reaction vessel 37 through piping 34. Next, an enzyme reaction substrate is added to the reaction vessel 37 from the enzyme reaction substrate adding device 31 through a feed pipe 35. By this addition, the reaction of an enzyme with the substrate proceeds to produce a substance having a property of luminescence or the like. In the apparatus shown in FIG. 2 (b), a sample delivery device 42 is arranged on the reaction vessel 37, and, for example, each reaction vessel 37 is delivered to a measuring device 33 at specified intervals of time; for example, the first reaction vessel 15 minutes after the addition of the enzyme reaction substrate, the second reaction vessel 20 minutes after the addition of the enzyme reaction substrate and the third reaction vessel 15 minutes after the addition of the enzyme reaction substrate are sent to the measuring device 33, and the accelerator is fed to each reactor vessel from the accelerator adding device 32 through piping 36. After a specified time (for example, two seconds) from the addition of the accelerator, the intensity of light emission or the like of the produced substance is measured by the measuring device 33, for example, a luminometer. Thus, the intensity of luminescence or the like for several periods of time of enzyme substrate reaction is measured, and by the above described technique the enzyme activity of the sample can be calculated.

The embodiment of the reaction unit 2 shown in FIG. 2 (c) comprises piping 40 for allowing the lysis treatment provided sample fluid from the collection unit A to flow, an enzyme reaction substrate adding device 31 for adding an enzyme reaction substrate to the lysis treatment provided sample fluid which flows in piping 40, an accelerator adding device 32 for adding an accelerator to the enzyme reaction substrate added sample solution, and a measuring device for measuring the quantity of luminescence or the like of a luminescent, fluorescent or color developing compound produced by the enzyme substrate reaction. The piping 40 is arranged within a thermostatic chamber 39 and is preferably maintained, for example, at 37° C.

The lysis treatment provided sample fluid from the collection unit A flows in the piping 40 and is added with an enzyme reaction substrate from the enzyme reaction substrate adding device 31. By this addition, the reaction of an enzyme with the substrate is allowed to proceed to produce a substance having a property of luminescence or the like. In the apparatus shown in FIG. 2 (c), the enzyme reaction substrate adding device 31 is connected to the piping at its three positions and, for example, each position of the piping to be connected to the enzyme reaction substrate adding device and the flow rate of the sample solution in the piping are adjusted such that the flowing time of the sample fluid from the first joint, the second joint and the third joint of the enzyme reaction substrate adding device 31 to the joint of the accelerator adding device come to 20 minutes, 15 minutes and 10 minutes, respectively. Further, each position of the joint of the accelerator adding device 32 and each flow rate of the sample fluid in the piping are adjusted such that the flowing time of the sample fluid from the joint of the accelerator adding device 32 to the measuring device 33 come to, for example, two seconds. After the sample fluid has reached all three joints of the enzyme reaction substrate adding device, the enzyme reaction substrate is simultaneously added to the three joints. Then, 10 minutes, 15 minutes and 20 minutes after the addition of the enzyme reaction substrate, an accelerator is added from the accelerator adding device 32, and two seconds later, the intensity of luminescence or the like is measured using the measuring device 33, and thus the intensity of luminescence or the like in the case of the enzyme substrate reaction of 10 minutes, 15 minutes and 20 minutes can be measured, and on the basis of the measured values, the enzyme activity of the samples can be calculated by the above explained technique.

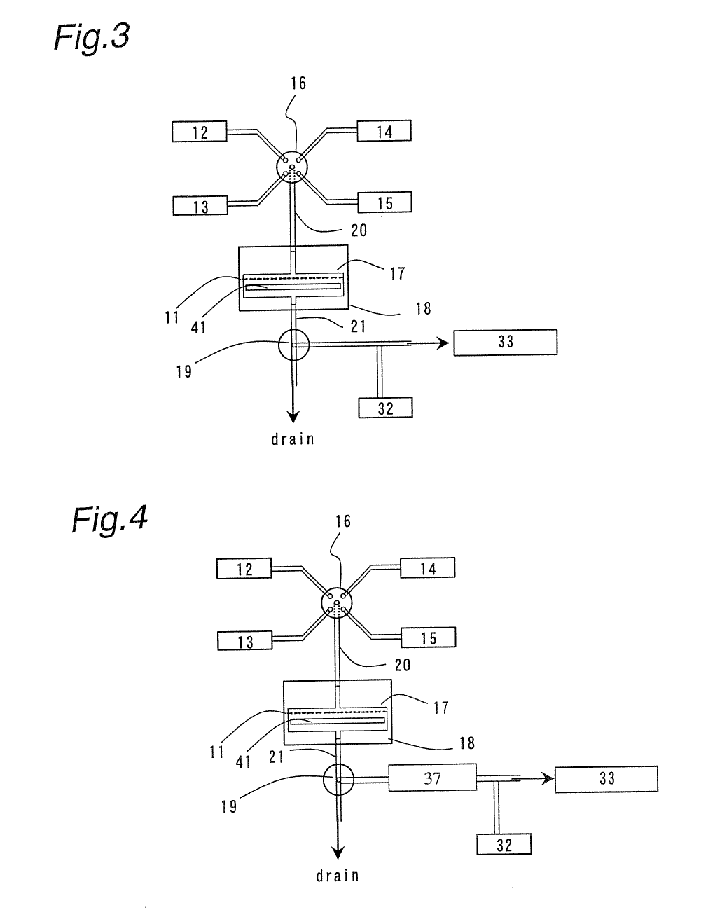

Further, the present invention relates to an apparatus for detecting bacteria (preferably E. coli and/or coliforms) comprising a collection/reaction vessel comprising a filter membrane having a pore diameter of 0.6 to 5.0 μm and/or a filter membrane having a flow rate of distilled water passed, measured in accordance with ASTM F317-72, of 50 to 500 mL/min·cm², a sample feed mechanism for feeding a sample fluid to the primary side of the filter membrane, an enzyme reaction substrate feed mechanism for feeding an enzyme reaction substrate and an enzyme activity measuring device for measuring an enzyme activity. The outline of the embodiment is shown in FIGS. 3 to 8.

In FIG. 3, the detection apparatus comprises a collection vessel 17 for housing a filter membrane 11 having a pore diameter of 0.6 to 5.0 μm and/or a filter membrane 11 having a flow rate of distilled water passed of 50 to 500 mL/min·cm² and a perforated plate 41 for rendering the flow rate of water passed of the filter membrane constant and an enzyme activity measuring device 33 for measuring an enzyme activity. The collection vessel 17 is arranged in a thermostatic chamber 18 and can be maintained at 30° C. to 45° C., preferably at 37° C. The collection vessel 17 is preferably equipped with a stirring device, a vibrating device (not shown in the Figure) or the like for sufficiently bringing the collected bacteria (such as E. coli) into contact with an enzyme reaction substrate fluid and a lysing agent. To the primary side of the filter membrane 11 in the collection vessel 17, a purging fluid feed mechanism 12 for feeding a purging fluid (air, pure water or the like) to purge the inside of the collection vessel 17, a sample fluid feed mechanism 13 for feeding a test fluid to the primary side of the filter membrane 11, an enzyme reaction substrate feed device 14 for feeding an enzyme reaction substrate and, if necessary, a lysing agent and a washing agent feed mechanism 15 for feeding a washing agent are connected through piping having a change-over valve 16 and constructed in such a manner that a desired amount of each fluid can be fed at a desired time. To the secondary side of the filter membrane 11 of the collection vessel 17, piping 21 having a three-way valve 19 is connected and constructed such a manner that the filtrate, excess washing solution and lysing agent and the like are discharged as a drain, and a sample fluid containing an enzyme reaction product (a dioxetane derivative) for measuring an enzyme activity is sent to the enzyme activity measuring device 33. To the piping 21, an accelerator adding device 32 is connected and the enzyme activity measuring sample after addition of a light emission accelerator or the like can be also sent to the enzyme activity measuring device 33. The enzyme activity measuring device 33 may be a known measuring device such as a luminometer having a photomultiplier tube and a photodiode and a CCD camera.

Now, the method of detecting bacteria using the detection apparatus shown in FIG. 3 will be explained. First, a test fluid is fed from the sample fluid feed mechanism 13 to the primary side of the filter membrane 11 at a constant flow rate. The test fluid is filtered with the filter membrane 11 to collect the bacteria (E. coli and the like) of the target for measurement on the filter membrane 11 and the filtrate is discharged as a drain through the piping 21 via a valve 19. Then, the change-over valve 16 is operated to change and a washing solution (for example, a solution containing a nonionic surfactant such as Triton X-100) is fed from the washing solution feed mechanism 15 to the primary side of the filter membrane 11 to perform washing by passing water. The washing solution passed through the filter membrane 11 is discharged as a drain through the piping 21 via the valve 19. Then, the change-over valve 16 is operated to change and a purging fluid (for example, air or pure water) is blown from the purging fluid feed mechanism 12 to purge the inside of the collection vessel 17, and the washing solution remaining on the filter membrane 11 is discharged as a drain through the piping 21. Then, the change-over valve 16 is operated to change and an enzyme reaction substrate fluid is fed from the enzyme reaction substrate feed mechanism 14 to the collection vessel 17. At this instance, the enzyme substrate fluid is preferably a mixture of an enzyme reaction substrate [for example, 3-(4-methoxyspiro-{1,2-dioxetan-3,2′-(5′-chloro)-tricyclo-[3.3.1.1^{3,7}]decan}-4-yl)phenyl-β-D-glucuronide or its derivative] and a lysing agent (for example, a nonionic surfactant such as Triton X100, an anionic surfactant such as sodium dodecyl sulfate (SDS)). The cell walls of the bacteria collected on the filter membrane 11 are lysed with the lysing agent to expose an enzyme. The enzyme reacts with the enzyme reaction substrate to produce an enzyme reaction product (for example, a dioxetane derivative) having a property of luminescence. Then, to this enzyme reaction fluid, a light emission accelerator (for example, Accelerator II: trade name, Tropix, Inc.) or the like for accelerating luminescence is fed from the acceleration feed mechanism 32 and brought into contact with the enzyme reaction fluid for a specified time (for example, within 10 seconds), and thereafter sent to the enzyme activity measuring device 33 such as a luminometer having a photodiode and a photomultiplier tube and a CCD camera, and the quantity of luminescence or the like is measured there. The enzyme activity can be calculated from the measured value of the quantity of luminescence or the like using the previously prepared working curve.

Now, several modified embodiments of the measuring device of the present invention will be shown in FIGS. 4 to 8. The apparatus shown in FIGS. 4 to 8 have basically the same construction as the construction of the apparatus shown in FIG. 3, and the same reference marks are given to the same constituting elements and their explanation is omitted, and different constructions will be explained.

In the embodiment shown in FIG. 4, a reaction vessel 37 is disposed downstream of the collection vessel 17. The reaction vessel 37 is installed in a thermostatic chamber (now shown in the Figure) and the temperature within the reaction vessel 37 is desirably maintained at 30° C. to 45° C., preferably 37° C. In this instance, it is unnecessary for the enzyme reaction to sufficiently proceed in the collection vessel 17, and accordingly the collection vessel 17 may or may not be positioned within the thermostatic chamber 18. The reaction vessel 37 which can be used may be loop-shaped piping which is installed in the thermostatic chamber 18. In order to bring an enzyme into good contact with an enzyme reaction substrate to accelerate the reaction, the reaction vessel 37 is preferably equipped with a stirring device or a vibrating device. A purging fluid (preferably air) may be sent from the purging fluid feed mechanism 12, and the sample fluid containing a target substance for measurement may be sent from the reaction vessel 37 to the measuring device 33. The embodiment shown in FIG. 4 has a reaction vessel 37 separate from the collection vessel 17, and accordingly, the extraction of an enzyme by lysis action can be mainly carried out in the collection vessel 17, and the reaction of the enzyme with the enzyme reaction substrate can be mainly advanced in the reaction vessel 37. Furthermore, there is an advantage of further improving the measuring sensitivity since the contact time between the enzyme of the bacteria (enzyme) and the enzyme reaction substrate is prolonged to advance the reaction.

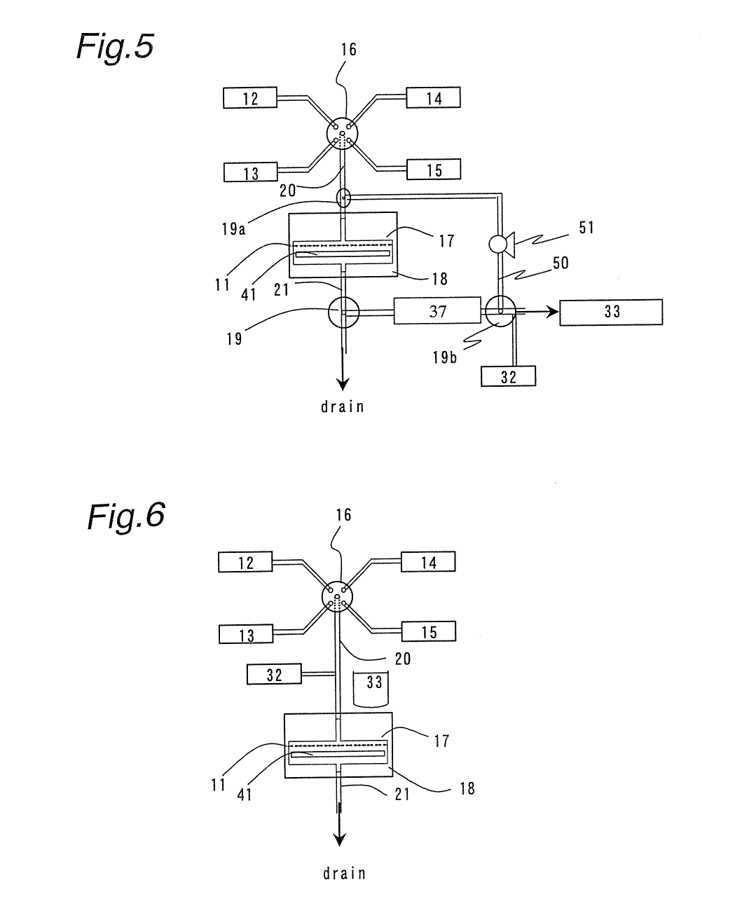

According to the embodiment shown in FIG. 5, a circulation line 50 and a circulation pump 51 are positioned downstream of the reaction vessel 37 and a sample fluid containing a substance of the target sample for measurement, an enzyme, an enzyme reaction substrate and the like is returned to the collection vessel 17. By recycling the sample fluid to the collection vessel 17, stirring of the sample fluid can be caused to accelerate the lysis of the bacteria remaining on the filter membrane 11, and the substance of the target sample for measurement, the enzyme, the enzyme reaction substrate and the like can be washed out into the sample fluid, and thus the contact time between the enzyme and the enzyme reaction substrate is prolonged to further advance the reaction, and there is an advantage that due to a rise in the concentration of the target substance for measurement, the measuring sensitivity can be improved and, simultaneously, the total amount of the enzyme reaction substrate added may be in a small amount.

According to the embodiment shown in FIG. 6, the collection vessel 17 is constructed by a light transmittable substance such as a transparent resin and transparent glass to utilize the collection vessel 17 as such as a measuring cell. The accelerator feed mechanism 32 is arranged upstream of the collection vessel 17 so as to feed an accelerator to the primary side of the filter membrane 11. Further, the enzyme activity measuring device 33 is arranged so as to measure the quantity of luminescence from the primary side of the filter membrane 11. In this embodiment, a test fluid, a lysing agent and an enzyme reaction substrate are fed to the primary side of the filter membrane 11 and the enzyme reaction is advanced to produce a target substance for measurement, and thereafter a light emission accelerator or the like is fed to accelerate luminescence, and the primary filtering surface of the filter membrane 11 is measured by the enzyme activity measuring device 33. In this embodiment, the enzyme reaction and the enzyme activity measurement can be carried in the collection vessel 17, and the apparatus construction and measuring operation can both be remarkably simplified.

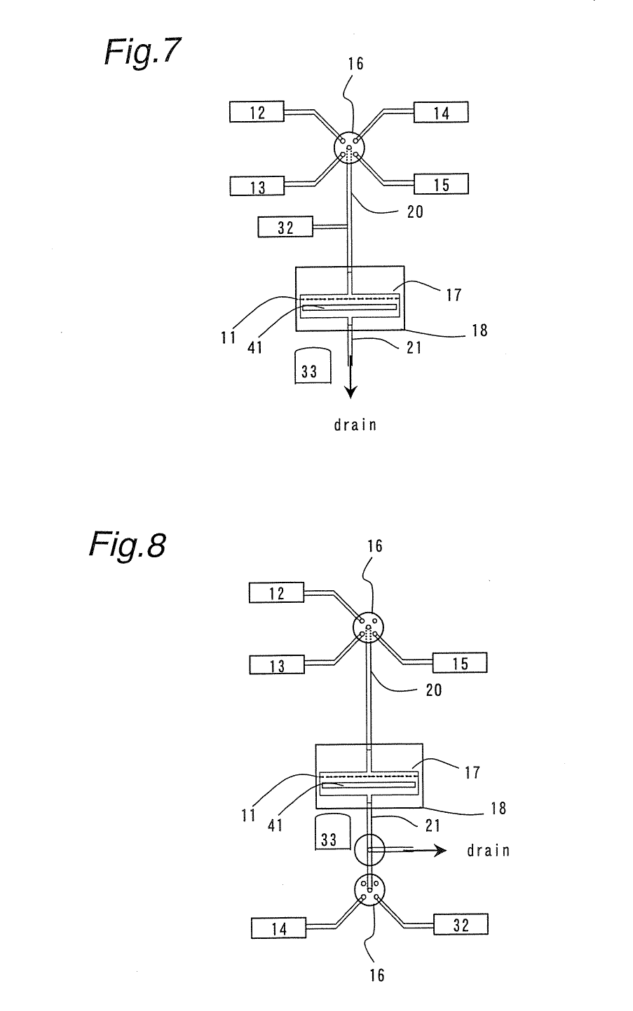

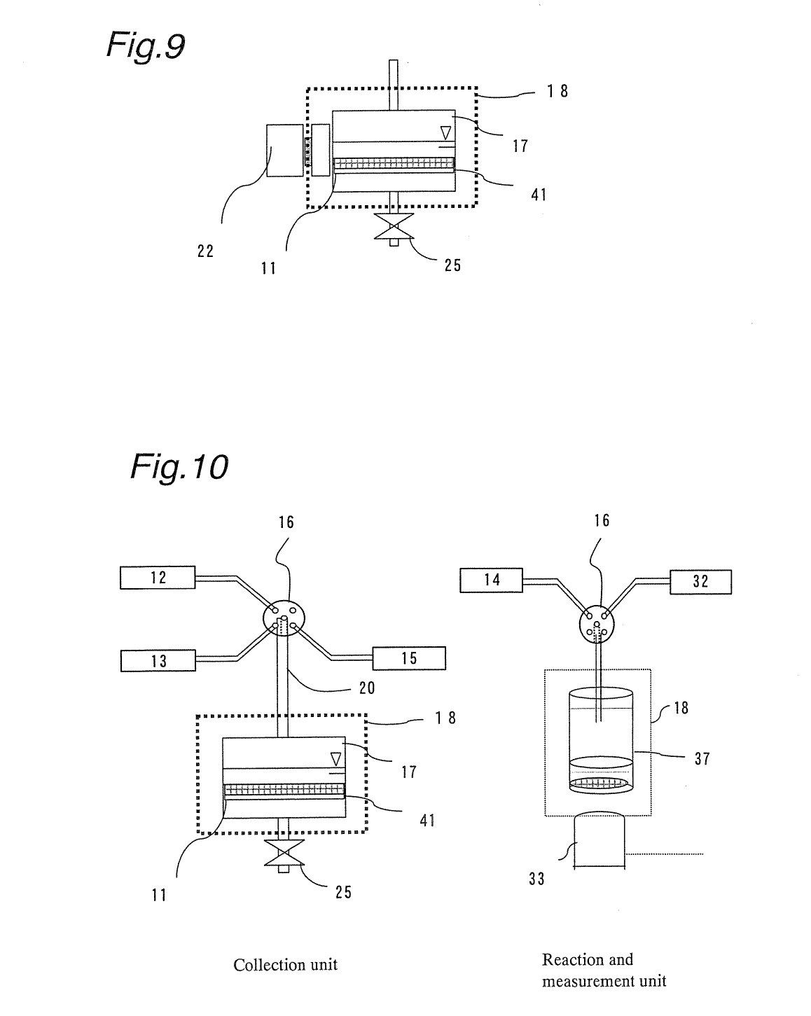

The embodiment shown in FIG. 7 is nearly the same as in FIG. 6 but the enzyme activity measuring device 33 is arranged so as to measure the secondary filtering surface of the filter membrane 11 in the collection vessel 17. In the measuring device of the type which does not circulate the reaction fluid in the enzyme reaction, it is preferred that a specified amount of space is provided on the primary side of a filter unit as in FIG. 9, and there the reaction substrate fluid is fed to allow enzyme lysis reaction to proceed while stirring the reaction unit. In this instance, the distance from the reaction fluid and the membrane to the measuring device 33 becomes long, to reduce the intensity of luminescence. Further, when the reaction fluid leaks during the enzyme reaction, the amount of the reaction fluid on the primary side varies to easily cause an error. On the other hand, on photometry from the secondary side, the distance to the sample is short and the surface of the membrane is directly subjected to photometry, and accordingly light reflects white filter paper to obtain higher intensity of luminescence.

According to the embodiment shown in FIG. 8, the test fluid and the washing solution are fed to the primary side of the filter membrane 11, and the enzyme reaction substrate and the accelerator are fed (injected) to the secondary side to measure the secondary filtering surface. The sample fluid feed mechanism 13 and the washing solution feed mechanism 15 are connected to the collection vessel 17 on the primary side of the filter membrane 11 through piping 20, and the enzyme reaction substrate fluid feed mechanism 14 and the accelerator feed mechanism 32 are connected to the collection vessel 17 on the secondary filtering surface of the filter membrane 11 through the piping 21 (20), and the enzyme activity measuring device 33 is arranged below the collection vessel 17. According to this embodiment, the piping 21 acts as a discharge line for discharging the test fluid and the washing solution from the collection vessel 17 and, simultaneously, acts as a feed line for feeding the enzyme reaction substrate fluid and the accelerator to the collection vessel 17. The bacteria of the target for measurement are collected on the filter membrane 11, and the enzyme reaction substrate and the accelerator fed (injected) from the secondary side of the filter membrane 11 permeate into the filter membrane 11 and move to the primary side of the filter membrane 11 and are brought into contact with the bacteria collected on the filter membrane 11.

FIG. 9 also shows a specific example of the collection vessel 17. The collection vessel 17 is positioned within the thermostatic chamber 18 and a shaker 22 for shaking the collection vessel 17 is fixed thereto. The filter membrane 11 and the perforated plate 41 are fixed within the collection vessel 17. The collection vessel 17 has a specified amount of space so as to mix the reaction fluid while holding the reaction fluid. Piping for feeding and discharging a fluid is fixed to the collection vessel 17 on the primary and secondary sides of the filter membrane 11, respectively. In the specific example shown in the Figure, a valve 25 is fixed to the piping on the secondary side to prevent the fluid from flowing out. However, as is shown in FIGS. 2 to 5, when a three-way valve is fixed to piping, the valve 25 may be unnecessary.

FIG. 10 shows one example of the detection device comprising a collection unit using a collection vessel 17 to which the filter membrane 11 is detachably fixed and a reaction/measuring unit. The collection unit comprises a collection vessel 17, a sample fluid feed mechanism 13 connected to the collection vessel 17 through piping and a change-over valve 16, a washing solution feed mechanism 15 and a purging fluid feed mechanism 12. The collection vessel 17 is constructed in an opening and closing manner in a suitable embodiment and can detachably fix the filter membrane 11. The reaction/measuring unit comprises the reaction vessel 37 positioned within the thermostatic chamber 18, the enzyme reaction substrate fluid feed mechanism 14 and the accelerator feed mechanism 32 which are connected to the reaction vessel 37 through the piping 20 and the change-over valve 16 and the enzyme activity measuring device 33 positioned so as to measure from the bottom of the reaction vessel 37. After bacteria are collected from the test fluid, the filter membrane 11 is taken out of the collection vessel 17 and transferred to the reaction vessel 37, and the enzyme reaction substrate fluid and the accelerator are added thereto and the quantity of luminescence or the like is measured by the enzyme activity measuring device 33.

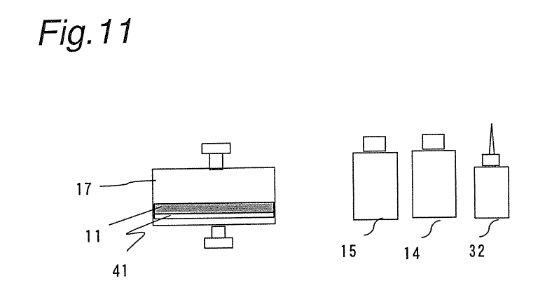

FIG. 11 shows an embodiment of a measuring kit. The measuring kit comprises a collection/reaction vessel 17, an enzyme reaction substrate fluid feed container 15, a washing solution feed container 14, an accelerator feed container 32. The collection/reaction vessel 17 comprises a filter membrane 11 having a pore diameter of 0.6 to 5.0 μm and/or a filter membrane 11 having a flow rate of distilled water passed of 50 to 500 mL/min·cm² and a perforate plate 41 for rendering the flow rate of water passed through the filter membrane 11 constant. It is preferred that the feed container for each fluid is constructed suitably for measuring collection of the fluid, for example, by using a syringe in such a manner that the cover is made of a silicone rubber and the needle of the syringe is pierced into a fluid to collect it or by using a dropper. The measuring kit is suitable for simple measurement by hand method.

The embodiments of the present invention are as follows.

1. A method of detecting bacteria comprising passing a sample fluid through a membrane having a pore diameter of 0.6 to 5.0 μm and/or a flow rate of distilled water passed of 50 to 500 mL/min·cm² to collect the bacteria in the sample fluid on the filter membrane, subjecting the bacteria collected on the filter membrane to lysis treatment to extract an enzyme and adding a predetermined enzyme reaction substrate to the extract fluid to allow enzyme substrate reaction to proceed, and measuring an enzyme activity to quantify the number of the bacteria in the sample fluid.

2. The method of Item 1, wherein a surfactant is passed through the bacteria collected filter membrane to wash the filter membrane, and thereafter the bacteria collected on the filter membrane is subjected to lysis treatment.

3. A method of detecting bacteria comprising passing a sample fluid through a membrane having a pore diameter of 0.6 to 5.0 μm and/or a flow rate of distilled water passed, measured in accordance with ASTM F317-72, of 50 to 500 mL/min·cm² to collect the bacteria in the sample fluid on the filter membrane, bringing the filter on which the bacteria is collected into contact with a fluid containing a lysing agent and an enzyme reaction substrate to allow enzyme reaction to proceed, measuring the enzyme activity to quantify the number of bacteria in the sample fluid.

4. The method of Item 3, wherein a surfactant is passed through the bacteria collected filter to wash the filter membrane, and thereafter the enzyme activity of the bacteria collected on the filter membrane is measured.

5. The method of Item 3 or 4, wherein in the method of measuring bacteria by measuring the enzyme activity derived from the bacteria collected on the filter membrane by the intensity of luminescence, the filter membrane is dipped in the enzyme substrate fluid in a reaction chamber having a bottom area not smaller than the filtering surface of the filter membrane to allow enzyme reaction to proceed, and the quantity of luminescence from the entire surface of the filter membrane is measured from its bottom surface.

6. The method of any one of Item 1 to Item 5, wherein the bacteria of the target for measurement are E. coli, coliforms and/or fecal coliforms, and the number of E. coli, coliforms and/or fecal coliforms in the sample fluid is quantified based on the enzyme activity of β-glucuronidase extracted from E. coli, coliforms and/or fecal coliforms.

7. The method of Item 6, wherein 3-(4-methoxyspiro{1,2-dioxetan-3,2′-(5′-chloro)-tricyclo-[3.3.1.1^{3,7}]decan}-4-yl)phenyl-β-D-glucuronide (Glucuron: registered trademark) or its derivative is used as the enzyme reaction substrate.

8. The method of any one of Item 1 to Item 5, wherein the bacteria of the target for measurement are coliforms and/or fecal coliforms and the number of coliforms and/or fecal coliforms in the sample fluid is quantified based on the enzyme activity of β-galactosidase extracted from the coliforms and/or fecal coliforms.

9. The method of Item 8, wherein 3-(4-methoxyspiro{1,2-dioxetan-3,2′-(5′-chloro)-tricyclo-[3.3.1.1^{3,7} ]decan}-4-yl)phenyl-β-D-galactopyranoside or its derivative is used as the enzyme reaction substrate.

10. An apparatus for detecting the bacteria in a sample fluid comprising a collection unit and a reaction unit for receiving a fluid to be fed from the collection unit to allow enzyme substrate reaction to proceed, wherein the collection unit is equipped with a filter for housing a filter membrane having a pore diameter of 0.6 to 5.0 μm and/or a filter membrane having a flow rate of distilled water passed of 50 to 500 mL/min·cm² and the collection unit comprises a sample feed device for feeding a sample fluid to the primary side of the filter membrane, a washing solution feed device for feeding a washing solution to the primary side of the filter membrane, a means to extract enzymes in the bacteria from the bacteria collected on the filter membrane and a means to send an enzyme extracted fluid obtained by extraction treatment to the reaction unit, and the reaction unit comprising an enzyme reaction substrate adding device for feeding an enzyme reaction substrate to the enzyme extract fluid from the collection unit, an accelerator adding device for adding an accelerator to the enzyme reaction substrate added reaction fluid, and a measuring device for measuring the quantity of luminescence, fluorescence or color developing of the accelerator added reaction solution.

11. An apparatus for detecting the bacteria in a sample fluid comprising a filter reaction unit comprising a filter for housing a filter membrane having a pore diameter of 0.6 to 5.0 μm and/or a filter member having a flow rate of distilled water passed of 50 to 500 mL/min·cm², a sample feed device for feeding a sample fluid to the primary side of the filter membrane, a washing solution feed device for feeding a washing solution to the primary side of the filter membrane, an enzyme reaction substrate adding device for feeding an enzyme reaction substrate and a thermostatic device for maintaining the enzyme reaction substrate fluid in the state of the filter membrane dipped therein, and a means to send the reaction solution after the enzyme substrate reaction to a measuring device, an accelerator adding device for adding an accelerator to the reaction fluid after the enzyme substrate reaction to be fed to the measuring device and a measuring unit for measuring the quantity of luminescence, fluorescence or color developing of the accelerator added reaction solution.

### EXAMPLES

Now, the present invention will be explained more specifically. The present invention is not to be limited by the following description.

### Example 1

### Relationship between Material and Diameter of Membrane Filter Used for Collecting E. coli and Collection Ratio of Foreign Matters Affecting E. coli and Background

In this Example, with the use of commercially available membrane filters (nitrocellulose membrane filter, cellulose acetate membrane filter and polycarbonate membrane filter) having various pore diameters, the relationship between among the pore diameter, the efficiency of collecting bacteria on each membrane and the background in the enzyme activity test was examined. The property values (values published by the manufactures) of each membrane filter used in the experiment are shown in Table 1. As can be understood from Table 1, the standards of membrane filters may be shown by pore diameter or flow rate of distilled water passed. In the examples, for convenience, they are expressed by pore diameter, and these numerals may be expressed in terms of the flow rate of distilled water passed using Table 1.

TABLE 1
Standard Performance of Membrane Filters (from
Catalogs issued by Manufactures)

| | Nitrocellulose Membrane Filter | | | | | | |
|---|---|---|---|---|---|---|---|
| Pore Diameter (μm)* | 5.0 | 3.0 | 1.0 | 0.8 | 0.65 | 0.45 | 0.2 |
| Porosity (%) | 81 | 81 | 80 | 80 | 79 | 78 | 73 |
| Thickness (μm) | 160 | 155 | 150 | 150 | 150 | 145 | 133 |
| Flow Rate** | 400 | 300 | 200 | 165 | 120 | 45 | 17.5 |
| B.P.*** | 5.8 × 10⁻² | 7.0 × 10⁻² | 9.6 × 10⁻² | 0.11 | 0.14 | 0.24 | 0.37 |

Cellulose Acetate Membrane Filter

| | | | | |
|---|---|---|---|---|
| Pore Diameter (μm)* | 3.0 | 0.8 | 0.45 | 0.2 |
| Porosity (%) | 78 | 72 | 68 | 66 |
| Thickness (μm) | 135 | 125 | 125 | 125 |
| Flow Rate** | 500 | 160 | 35 | 16 |
| B.P.*** | 3.4 × 10⁻² | 6.8 × 10⁻² | 0.17 | 0.25 |

Polycarbonate Membrane Filter

| | | | | |
|---|---|---|---|---|
| Pore Diameter (μm)* | 1.0 | 0.8 | 0.4 | 0.2 |
| Thickness (μm) | 9 | 9 | 10 | 10 |
| Flow Rate** | 170 | 130 | 50 | 15 |
| B.P.*** | 3.2 × 10⁻² | 4.8 × 10⁻² | 8.2 × 10⁻² | 0.13 |

*Nominal value of the average pore diameter (tolerance range ±30%) published by the makers.
**Flow rate (mL/min · cm²) when 25° C. distilled water filtered with a membrane filter having a pore diameter of 0.10 μm was filtered under a reduced pressure of −0.069 MPa.
***Bubble point, Pressure (MPa) up to continuous bubbles coming from a completely wet sample (membrane filter). In the case of nitrocellulose and cellulose acetate as the materials, the bubble points were measured with water, and in the case of polycarbonate as the material, the bubble points was measured using isopropyl alcohol.

The efficiency of collecting bacteria of each of the above described membrane filters was examined. A sample was prepared by diluting sewage inflow water to 1/50 and passed through a diameter of 47 mm membrane filter. The flow rate of water passed through the filter having a pore diameter of 0.2 μm was set at 50 mL, that passed through the filter having a pore diameter of 0.40 to 0.45 μm was set at 100 mL and that passed through the filter having a pore diameter of 0.65 μm or more was set at 200 mL. The sample before filtration and the filtrate were subjected to colony test, and bacteria [GALase(+)] having β-galactosidase activity and bacteria [GLUase(+)] having β-glucuronidase activity in the sample and filtrate were counted with the use of Petrifilm (registered trademark, 3M). Specifically, with the use of “E-coli/coliforms measuring EC plate”, the bacteria in the sample and filtrate were incubated at 37° C. for 20 hours, and thereafter red colonies formed were counted as GALase(+) and blue colonies formed were counted as GLUase(+).

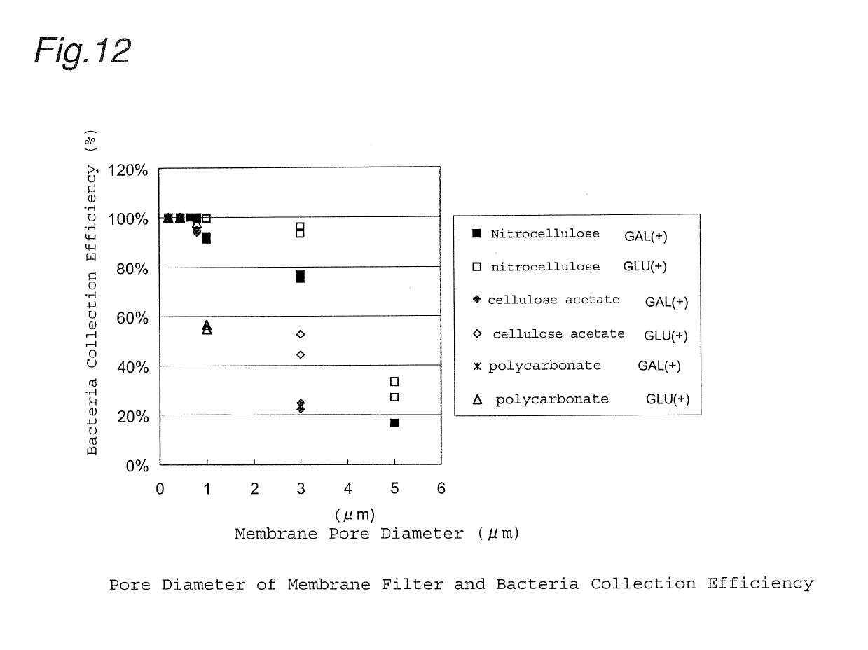

GALase(+) in the sample before filtration was 3,600 CFU/mL and GLUase(+) was 360 CFU/mL. The results of counting GALase(+) and GLUase(+) in the filtrate are shown in Table 2 and FIG. 12.

TABLE 2
Results of Counting GALase(+) and GLUase(+) in Filtrate

| | | Pore Diameter of Membrane (μm) | | | | | | |
|---|---|---|---|---|---|---|---|---|
| | | 0.2 | 0.40-0.45 | 0.65 | 0.8 | 1.0 | 3.0 | 5.0 |
| Material of Membrane | | Number of Bacteria (CFU/mL) | | | | | | |
| Nitrocellulose | GALase(+) | 0.1 | 0.4 | 1.6 | 22 | 300 | 870 | 3,000 |
| | GLuase(+) | 0.0 | 0.0 | 0.0 | 0.2 | 1.7 | 19 | 250 |
| Cellulose | GALase(+) | 3.6 | 0.2 | —* | 200 | —* | 2,800 | —* |
| Acetate | GLuase(+) | 0.1 | 0.0 | —* | 20 | —* | 190 | —* |
| Polycarbonate | GALase(+) | 0.9 | 0.5 | —* | N/A** | N/A** | —* | —* |
| | GLuase(+) | 0.1 | 0.0 | —* | 8 | 160 | —* | —* |

All numerical values show the mean values calculated by taking measurements twice.
*— shows that no test was carried out because commercial products did not exist or could not be obtained.
**N/A shows that due to a large amount of leaked bacteria, accurate counting could not be performed.

With the cellulose acetate having a membrane thickness of 100 μm or more and low adsorbability, bacteria started leaking from the pore diameter of 0.8 μm to lower the collection efficiency of bacteria with increased pore diameters but bacteria were collected in the form of E. coli entering the network structure, and accordingly even pore diameters of 1 μm or more used were high in the collection efficiency of bacteria and were within the range for practical purposes.

With the nitrocellulose having a membrane thickness of 100 μm or more and high adsorbability, bacteria started leaking from the pore diameter of 1.0 μm but the collection efficiency of bacteria was hardly lowered even with increased pore diameters. Due to its high adsorbability, the collection efficiency was higher than that of the cellulose acetate having the same network structure.

With the polycarbonate having a membrane thickness as small as 10 μm or less and low adsorbability, bacteria started leaking from the pore diameter of 0.8 μm and the collection efficiency of bacteria was rapidly lowered at the pore diameter of 1 μm. Thus, in the case of using polycarbonate, it was found that the use of the pore diameter of 0.8 μm or less was preferred.

From the above described results, it has been found that the use of a membrane filter having a pore diameter (an average pore diameter) of 0.6 to 5.0 μm, preferably 0.8 to 3.0 μm enables the collection of E. coli and the like within a range for practical purposes in high yield.

Further, it has been found that in the case of using filters having a membrane thickness of 10 μm or less, with pore diameters of 0.8 μm or less, E. coli and the like can be collected within a range for practical purposes in high yield.

Next, an experiment to eliminate foreign matters which adversely affect the lower limit value of quantification by raising the background of the enzyme activity test was carried out using the above described membrane filters. Using environmental water which was previously confirmed to be E. coli-free by the measurement using the above described Petrifilm (registered trademark), the change due to the difference in pore diameter and material was examined. The experiment was carried out by using the above used nitrocellulose membrane filter, cellulose acetate membrane filter and polycarbonate membrane filter.

As the enzyme reaction substrate solution to β-glucuronidase, a solution of 1 vol. % Glucuron (registered trademark), 0.1 M sodium phosphate and 10 mM EDTA was used.

The environmental water confirmed to be E. coli-free was dispersed in an ultrasonic bath and suction-filtered with filter paper No. 5A.

The obtained filtrate was first passed through a membrane filter having a pore diameter of 3.0 μm. Then, 50 mL of a solution containing 0.02 vol. % Triton X-100 was passed through the filter to perform filter washing. The filtrate and the washed water were combined, and then passed through a membrane filter having a pore diameter of 0.8 μm. Subsequently, 50 mL of a solution containing 0.02 vol. % Triton X-100 was passed through the filter to perform filter washing. Thereafter, the filtrations with membrane filters having a pore diameter of 0.45 μm and 0.2 μm were performed using the same procedure.

Collected substances by the filter having each pore diameter were subjected to β-glucuronidase enzyme activity test to examine the β-glucuronidase activity.

As the enzyme extracting solution, a solution obtained by adding 0.2 vol. % Triton X-100 to a 0.1 M sodium phosphate solution containing 0.1 wt. % BSA was used, and after washing with the above described 0.02 vol. % Triton X-100 solution, the filter having each pore diameter was dipped in this extracting solution to perform lysis treatment at 37° C. for 30 minutes.

As the reaction substrate solution for β-glucuronidase, a solution containing 1 vol. % Glucuron (registered trademark) 0.1 M sodium phosphate and 10 mM EDTA was used, and 180 μL of this reaction substrate solution was mixed with 100 μL of the enzyme extracted solution obtained in the above described lysis treatment. The resulting mixture was shaken at 37° C. for 5 to 30 minutes with the use of a thermostatic shaking machine. At the time of performing shaking for 5 minutes, 10 minutes, 15 minutes and 20 minutes, respective sample solutions were collected and 300 μL of a light emission accelerator [Accelerator II (trade name, Tropix, Inc.)] was added thereto to measure the quantity of luminescence after two seconds. The measurement of the quantity of luminescence was performed using a luminometer (LB-9507, manufactured by Berthold Technology).

The relative light unit (RLU) versus the shaking time (enzyme substrate reaction time) in each sample was plotted on a graph, and from the gradient (RLU/min) of the obtained straight line, the enzyme activity (β-glucuronidase activity) was found.

Since the test sample used in this example is a sample confirmed to be E. coli-free, the enzyme activity measured in each test can be thought to all be the background by foreign matters which adversely affect the enzyme activity test.

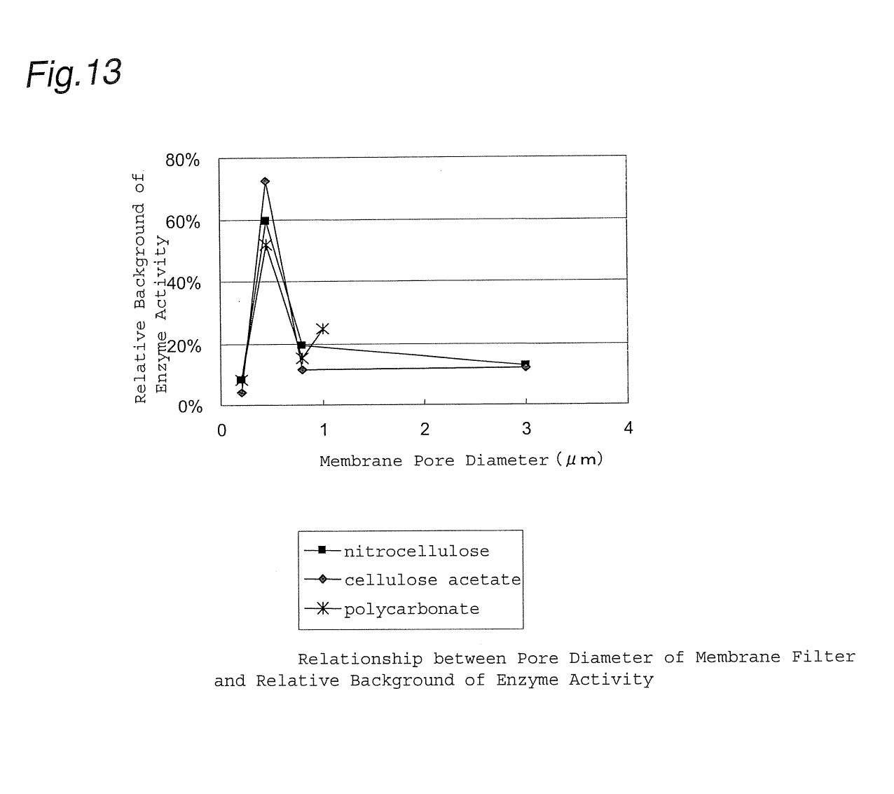

The β-glucuronidase activity (relative backgrounds) for the collected substances by each type of the membrane filters having respective pore diameters are shown in FIG. 13. The enzyme activity (background) measured by performing the same enzyme activity test for the sample before filtration was taken as 100% and the relative value of the enzyme activity to this value was taken as a relative background.

In the test with any membrane having a pore diameter of 0.4 to 0.45 μm, 50% or more of the background was detected. From FIG. 13, it can be understood that by the filtration with membrane filters having a pore diameter below 0.6 μm, foreign matters which adversely affect the enzyme activity test are all collected on the filter membranes to raise the background.

It has been found as shown in FIG. 13 that in order to eliminate the effect of foreign matters, the pore diameter is required to be not smaller than 0.6 μm. Further, it has been found from FIG. 12 that in order to collect E. coli with sufficient efficiency, the pore diameter is required to be 5.0 μm or less, particularly 3.0 μm or less. Putting together these results, it is shown that by filtering a sample fluid using a membrane filter having a pore diameter of 0.6 μm to 5.0 μm, preferably 0.8 μm to 3.0 μm to collect bacteria on the filter membrane and performing enzyme activity test for the collected bacteria, the adverse effect of the background can be eliminated to detect/quantify the bacteria in the sample with a practical range of accuracy.

Further, with the polycarbonate having a membrane thickness as thin as 10 μm or less and a low adsorbability, the pore diameter necessary to collect E. coli in high yield is not greater than 0.8 μm and the pore diameter for detecting the background is 0.4 to 0.45 μm, and accordingly, the range of pore diameters for obtaining an optimum washing effect is narrower than that of the thicker nitrocellulose membrane filter and cellulose acetate membrane filter. Thus, it has been found that a filter which is used in collecting E. coli preferably has a thickness of more than 10 μm.

### Example 2

### Rapid/High Sensitivity Measurement of E. coli

First, it is shown that by using Glucuron (registered trademark) as the enzyme reaction substrate for measuring β-glucuronidase activity, the E. coli measurement using the standard sample is rendered rapid/highly sensitive.

The number of E. coli in first settling basin overflow collected in a sewage treatment plant was found by the culture method (the colony counting method). Petrifilm (registered trademark) “E-coli/coliforms measuring EC plate”, a product of 3M, was used as the medium. The sample was cultured at 37° C. for 20 hours, and thereafter allowed to react with X-glu of an enzyme substrate specific to β-glucuronidase to form blue colonies and the gas generating blue colonies were counted as E. coli. This first settling basin overflow was suitably added to sterile diluting water to prepare seven test samples of standard samples having E. coli in the range 1×10¹ to 1×10⁴ CFU/mL. The first settling basin overflow contains 1×10⁶ CFU/mL of E. coli, and by diluting this overflow with sterile diluting water so as to reduce the number of E. coli in the range of 1×10¹ to 1×10⁴ CFU/mL, the amount of foreign matter co-present in the samples comes to 1/100 or less and the effect of measurement on the background can be eliminated.

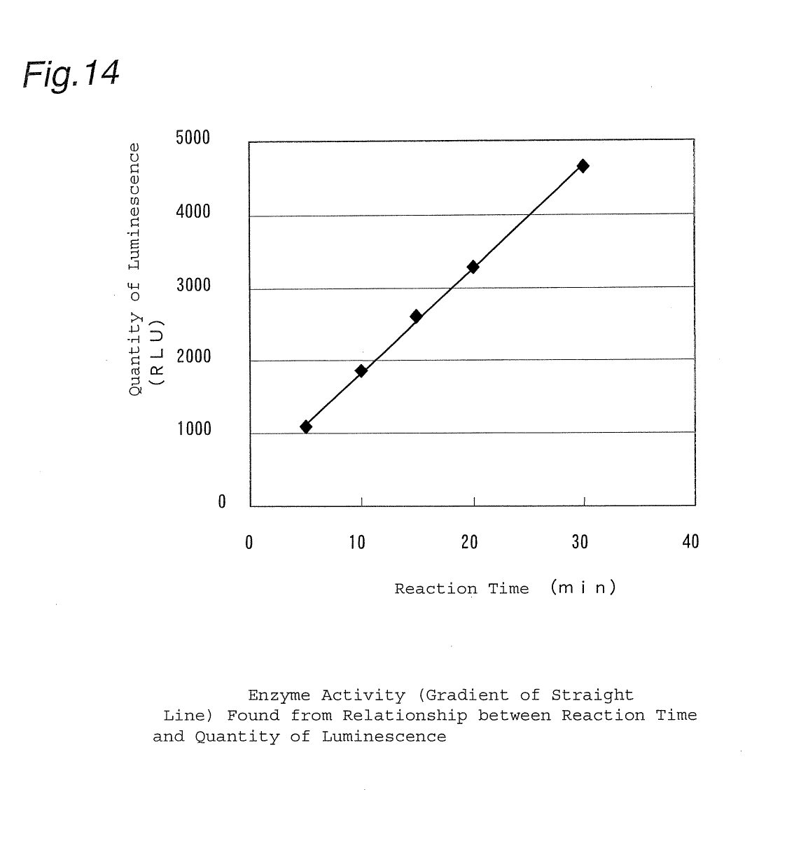

These standard samples were dispersed in an ultrasonic bath, and then suction-filtered with filter paper No. 5A. A concentrated lysing solution adjusted so as to have a BSA concentration of 0.1 wt. %, a concentration of a sodium phosphate solution of 0.1 M and a concentration of Triton X-100 of 0.2 vol. % after the addition/mixing of the samples was added to the samples to perform lysis treatment at 37° C. for 30 minutes. As the enzyme reaction substrate solution to β-glucuronidase, 100 μL of the supernatant was collected and mixed with 180 μL of a solution comprising 1 vol. % Glucuron (registered trademark), 0.1 M sodium phosphate and 10 mM EDTA. The obtained mixture was shaken with the use of a thermostatic shaker. After respective shaking times of 5 minutes, 10 minutes, 15 minutes and 20 minutes, respective samples were collected and 300 μL of a light emission accelerator [Accelerator II (trade name, Tropix, Inc.)] was added thereto to measure the quantity of luminescence after two seconds. The measurement of the quantity of luminescence was performed with the use of a luminometer (LB-9507, manufactured by Berthold Technology). The relative light unit (RLU) versus the shaking time (enzyme substrate reaction time) was plotted on a graph and the gradient of the obtained curve was taken as an enzyme activity (β-glucuronidase activity). In FIG. 14, one example of the curve of the relative light unit (RLU) versus the shaking time (enzyme substrate reaction time) prepared as described above is shown. In the curve of FIG. 14, the enzyme activity (the gradient of the straight line) was 141.9 RLU/min.

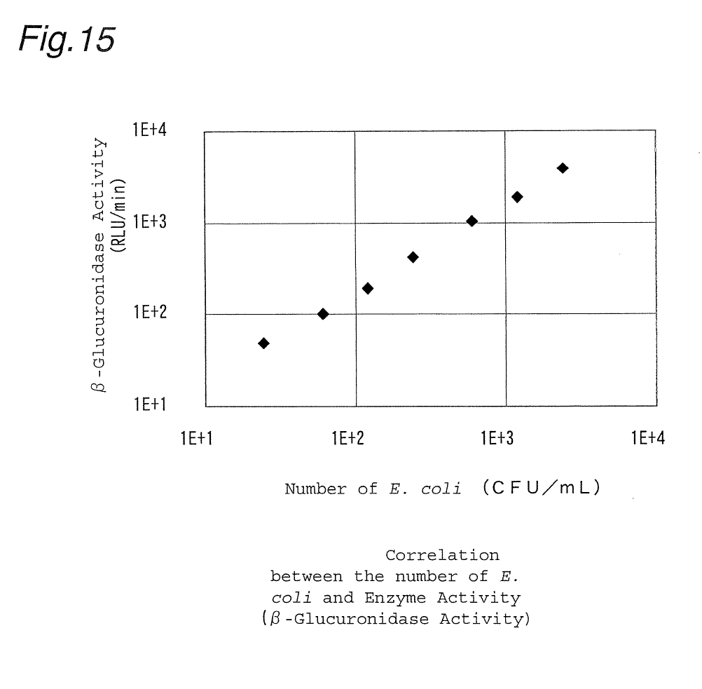

The correlation between the number of E. coli (colony count value) and the β-glucuronidase activity found in the above described technique is shown in FIG. 15.

As is shown in FIG. 15, a very superior linear relation was obtained in the range of the number of E. coli of 2.5×10¹ CFU/mL to 2.5×10³ CFU/mL. The lower limit value of quantification of E. coli was 40 times improved from 1.0×10³CFU/mL of the conventional method (method disclosed in George, I., Crop, P., and Servais, P., “Use of β-D-galactosidase and β-D-glucuronidase activities for quantitative detection of total and fecal coliforms in wastewater”, Can., J. Microbiol., 47, 670-675 (2001)).

Next, it is shown that by using a membrane filter, even in the case of using an actual sample containing foreign matters which affect the background of measurement, the same high sensitivity as the above described standard samples can be obtained.

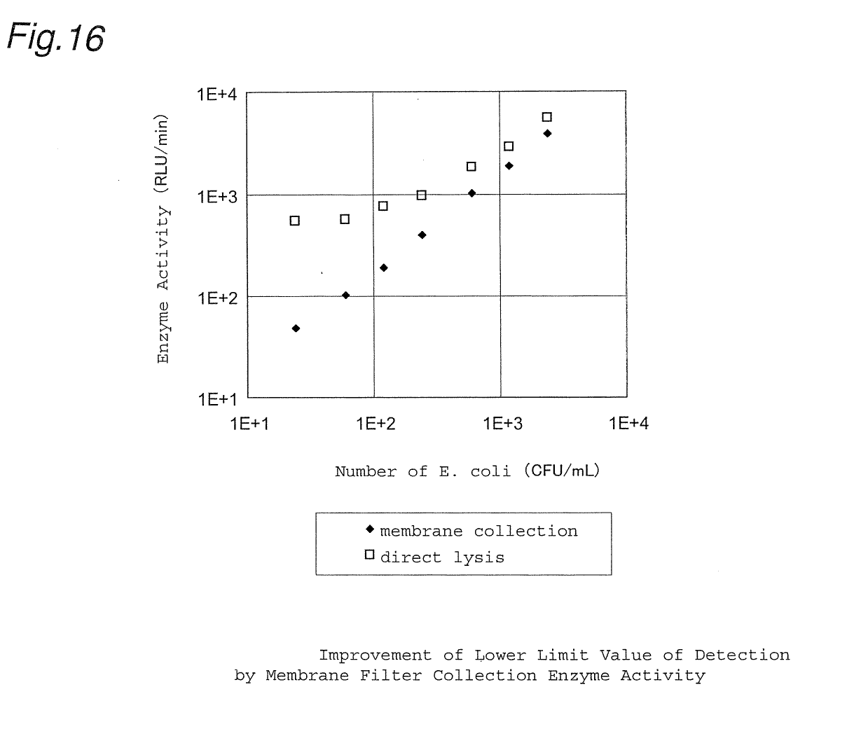

First settling basin overflow, secondary treatment water and aerobic basin outlet water collected from a sewage treatment plant were dispersed in an ultrasonic bath and then suction-filtered with filter paper No. 5A. The obtained filtrate was suitably mixed to prepare seven test samples having a number of E. coli in the range of 10¹ to 10⁴ CFU/mL. These samples were each divided into two groups and one group was passed through a membrane made of nitrocellulose having a pore diameter of 0.8 μm to collect E. coli on the membrane, and the collected E. coli was washed with 50 mL of the same solution containing 0.02 vol. % Triton X-100 as used in Example 1, and thereafter dipped in a solution obtained by adding 0.2 vol. % Triton X-100 to a 0.1 M sodium phosphate solution containing 0.1 wt. % BSA to lyse E. coli. The other group of the sample solutions was mixed with a solution obtained by adding 0.2 vol. % Triton X-100 to a 0.1 M sodium phosphate solution containing 0.1 wt. % BSA at a volume ratio of 1:1 without passing through the membrane filter to lyse E. coli and an enzyme extract fluid was obtained (the conventional method). For respective enzyme extract solutions, the same β-glucuronidase activity test as used in Example 1 was carried out to find an enzyme activity (RLU/min) and the actual E. coli detection/quantification test by colony test was carried out to examine the correlation between the number of E. coli and the enzyme activity. The result is shown in FIG. 16.

Since the E. coli in the sample solution was directly lysed in the conventional method, soluble foreign matters were mixed in the reaction solution to lower the accuracy of analysis due to the background noise, and the lower limit value of detection was increased. In contrast to this, according to the present invention, the E. coli was collected on the filter membrane, washed with a surfactant, and thereafter the enzyme activity test was carried out, and accordingly foreign matters were eliminated to improve the lower limit value of detection by one digit as shown in FIG. 16.

### Example 3

### Improvement of Sensitivity by Surfactant Washing

In this Example, the advantage of carrying out lysis treatment after washing the bacteria collected with a filter membrane with a surfactant and the optimum conditions in this instance will be examined.

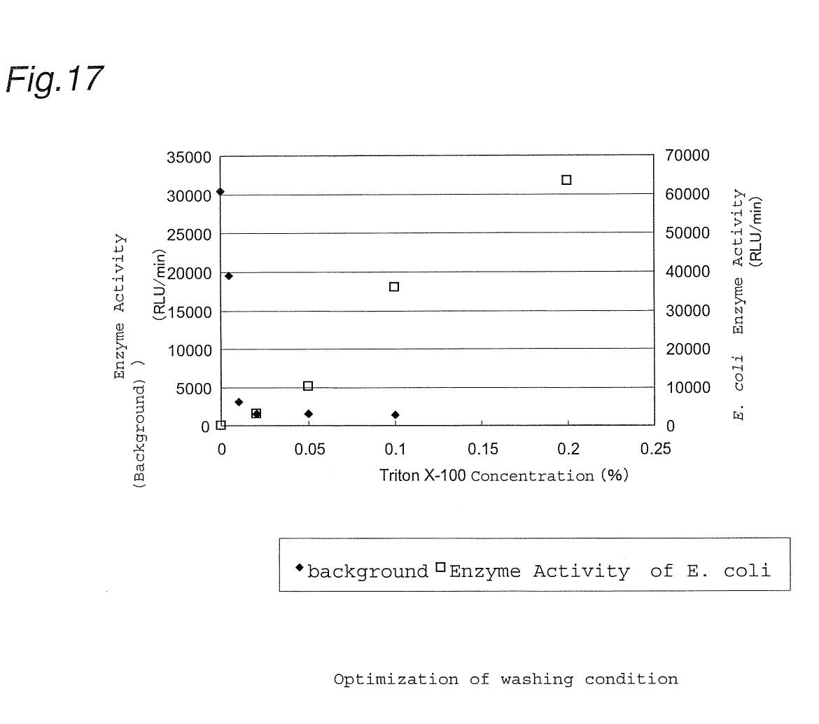

First, the effect of the reduction in background by washing the bacteria collected with a filter membrane with a surfactant was confirmed. Environmental water which had been previously confirmed to be E. coli-free by the measurement using the above described Petrifilm (registered trademark) was dispersed in an ultrasonic bath and then suction-filtered using filter paper No. 5A. The filtrate was passed through a nitrocellulose membrane filter having a pore diameter of 0.8 μm. Six test samples were prepared in the same manner. Through the membrane filter after passing the filtrate, respective solutions (washing solutions) containing 0 vol. %, 0.005 vol. %, 0.01 vol. %, 0.02 vol. %, 0.05 vol. % and 0.1 vol. % Triton X-100 were passed to wash the filter, and thereafter the β-glucuronidase activity to the remaining substances on the filter was found using the same procedure as in Example 1. The results are shown in FIG. 17.

Next, the loss of E. coli by washing with a surfactant was examined. From first settling basin overflow in a sewage treatment plant, a sample having high concentration E. coli was collected and diluted with sterile diluting water to prepare an E. coli standard sample. This sample was dispersed in an ultrasonic bath and then suction-filtered with the use of filter paper No. 5A. The filtrate was passed through a nitrocellulose membrane filter having a pore diameter of 0.8 μm to collect E. coli. Five test samples were prepared in the same manner. The E. coli collected membrane was impregnated with each of the solutions containing 0 vol. %, 0.02 vol. %, 0.05 vol. %, 0.1 vol. % and 0.2 vol. % Triton X-100 and left to stand in the state of the membrane impregnated for 5 minutes, and thereafter the β-glucuronidase activity (E. coli enzyme activity) was found using the same procedure as in Example 1. This enzyme activity was evaluated as the amount of losable E. coli by washing. The results are shown in FIG. 17.

It has been found as shown in FIG. 17 that in order to obtain sufficient effect of the reduction in the background, the concentration of Triton X-100 is required to be not less than 0.01 vol. %. On the other hand, with the concentrations of Triton X-100 of not less than 0.1 vol. %, losable E. coli increased. Thus, it has been found that the concentration of Triton X-100 of the washing solution for obtaining optimum washing conditions is 0.01 to 0.1 vol. %.

### Example 4

### Lysis Treatment with Ultrasonic Wave

In the present invention, the operation (lysis treatment) of extracting β-glucuronidase from E. coli may be performed with the use of a surfactant or ultrasonic wave. Even in the case of using ultrasonic wave, the addition of a surfactant to the reaction fluid is preferred in order to improve sensitivity.

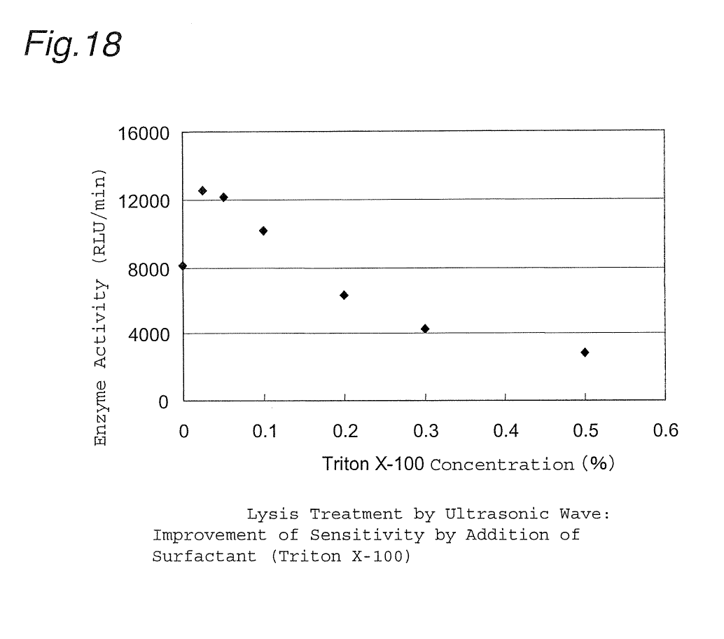

Secondary treatment water collected from a sewage treatment plant was dispersed in an ultrasonic bath and then suction-filtered with the use of filter paper No. 5A. The filtrate was passed through a membrane having a pore diameter of 0.8 μm to collect E. coli. The E. coli collected membrane was crushed by ultrasonic wave under the conditions of an AMP of 100 watt and a pulse of 1 second and a timer of 1 minute to extract β-glucuronidase (lysis treatment). One hundred microliters of the supernatant was collected, and Triton X-100 of a surfactant was added thereto so as to render its final concentration 0 vol. %, 0.025 vol. %, 0.05 vol. %, 0.1 vol. %, 0.3 vol. % and 0.5 vol. %, and thereafter the resulting mixture was mixed with 180 ML of the same enzyme reaction substrate solution as in Example 1. By the same procedure as in Example 2, the resulting mixture was shaken at 37° C. for a predetermined time using a shaker, and then 300 μL of a light emission accelerator was added thereto to cause luminescence and the quantity of luminescence was measured to find an enzyme activity. The relationship between concentration of a surfactant and the enzyme activity is shown in FIG. 18.

It is shown that in the case of finding an enzyme activity by carrying out the lysis treatment by ultrasonic wave crushing, the addition of a surfactant to the sample after the ultrasonic treatment improves the sensitivity by up to 60%. Further, it is understood that in the case of adding a surfactant for this purpose, as its amount added, the surfactant concentration in the solution preferably is 0.01 to 0.1 vol. %.

### Example 5

### Optimum Condition in Lysis with Surfactant Lysing Time

In extracting β-glucuronidase (lysis treatment) with the use of a surfactant, a specified lysing time is necessary.

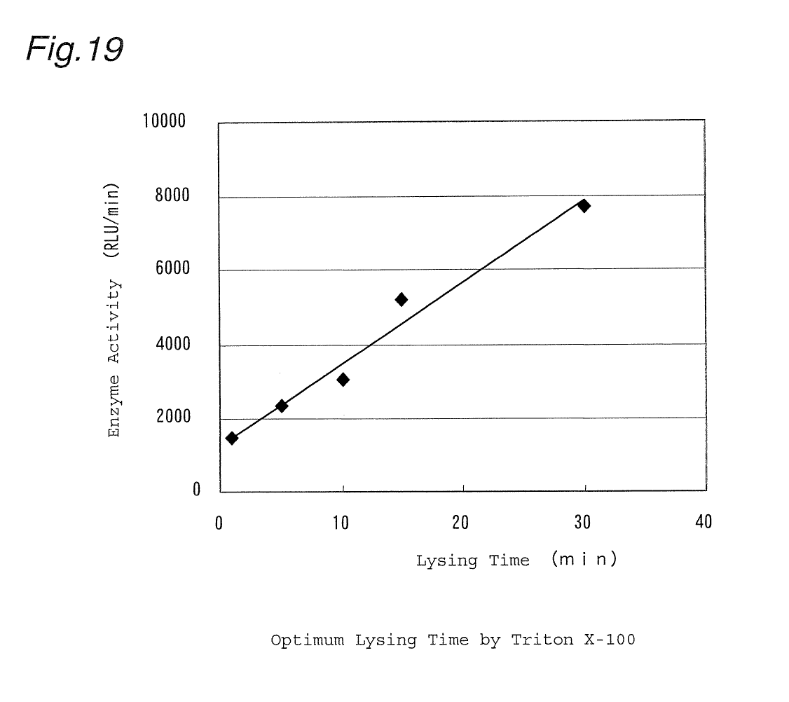

Secondary treatment water collected from a sewage treatment plant was dispersed in an ultrasonic bath and then suction-filtered with the use of filter paper No. 5A. The filtrate was passed through a membrane having a pore diameter of 0.8 μm to collect E. coli. As an enzyme extracting solution, a solution obtained by adding 0.1 vol. % Triton X-100 to a 0.1 M sodium phosphate solution containing 0.1 wt. % BSA was used, and the E. coli collected membrane was dipped in this extracting solution to perform lysis treatment at 37° C. for 0 minutes, 5 minutes, 10 minutes, 15 minutes and 30 minutes. One hundred microliters of the supernatant was collected and mixed with 180 μL of the above described enzyme reaction substrate solution. By the same procedure as in Example 2, the resulting mixture was shaken at 37° C. for a predetermined time using a thermostatic shaker, and then 300 μL of a light emission accelerator was added to cause luminescence and the quantity of luminescence was measured to find an enzyme activity. The relationship between the lysing time and the enzyme activity was shown in FIG. 19.

According to the conventional method disclosed in Japanese Patent Publication A No. 2000-270894, a surfactant and an enzyme reaction substrate were simultaneously added to a test sample. According to this method, the enzyme reaction was initiated in the state of insufficient progression of lysis reaction, and accordingly variations of the amount of the enzyme extracted and the concentration of the substrate became large, and sufficient analytic accuracy could not be obtained. On the other hand, in the method of the present invention, the reaction conditions became constant by adding the enzyme reaction substrate after sufficient lysis of E. coli and analytical accuracy was remarkably increased.

### Example 6

### Optimum Condition in Lysis with Surfactant: Concentration of Surfactant

In extracting β-glucuronidase (lysis treatment) by lysis using a surfactant, it is preferred to increase the concentration of Triton X-100. In this Example, the relationship between the efficiency of lysis by a surfactant and the inhibition of enzyme reaction was examined.

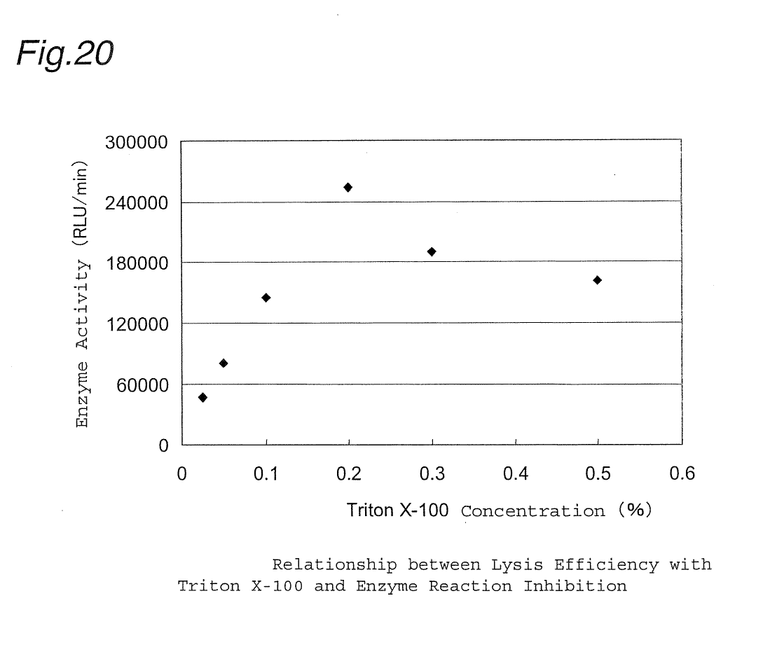

Secondary treatment water collected from a sewage treatment plant was dispersed in an ultrasonic bath and then suction-filtered using filter paper No. 5A. The filtrate was passed through a membrane having a pore diameter of 0.8 μm to collect E. coli. As enzyme extracting solutions, solutions obtained by adding 0 vol. %, 0.025 vol. %, 0.05 vol. %, 0.1 vol. %, 0.3 vol. % and 0.5 vol. % Triton X-100 to a 0.1 M sodium phosphate solution containing 0.1 wt. % BSA were used, and the E. coli collected membrane was dipped in these extracting solutions to perform lysis treatment at 37° C. for 30 minutes. One hundred microliters of the supernatant was collected and mixed with 180 μL of the above described enzyme reaction substrate solutions. By the same procedure as that of Example 2, the resulting mixture was shaken at 37° C. for a predetermined time with the use of a thermostatic shaker, and then 300 μL of a light emission accelerator was added thereto to cause luminescence and the quantity of luminescence was measured to find an enzyme activity. The relationship between the concentration of Triton X-100 and the enzyme activity is shown in FIG. 20. It has been found from the relationship between the efficiency of lysis and the inhibition of enzyme reaction that the optimum concentration of Triton X-100 for obtaining highest enzyme activity is around 0.2 vol. %. According to the conventional method disclosed in Japanese Patent Publication A No. 2000-270894, in the case of using Triton X-100 as a surfactant, its concentration is set at 0.1 vol. %. It is understood from the results of FIG. 20 that by setting the concentration of Triton X-100 at 0.2 vol. %, the sensitivity is improved by about two times. Further, it is understood from FIG. 20 that the concentration of the surfactant as a preferred lysing agent is 0.15 to 0.5 vol. %.

### Example 7

In this Example, the effect of the concentration of a surfactant as a lysing agent when the surfactant was added together with an enzyme reaction substrate was examined.

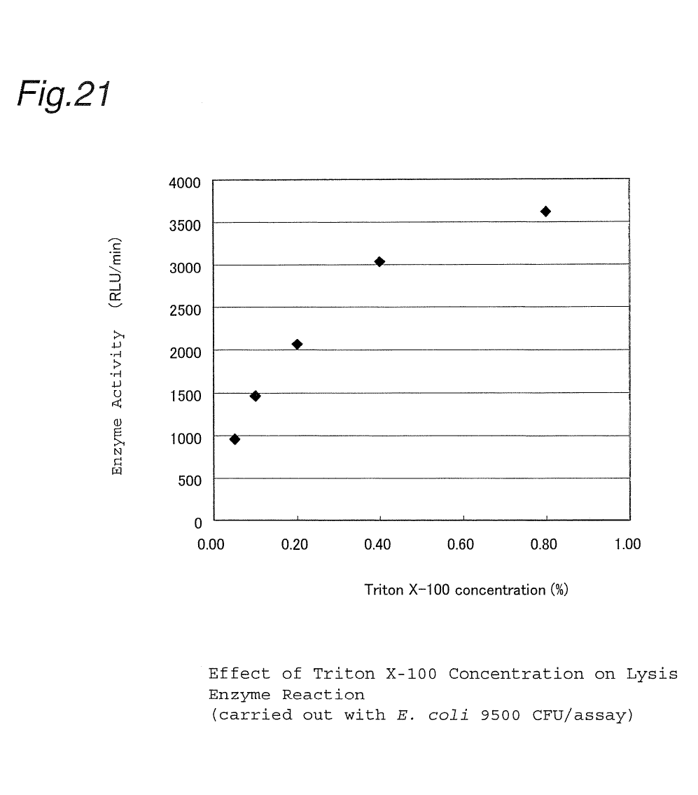

In an ultrasonic bath, 10 mL of sewage diluted with pure water to 1/10 was dispersed and then suction-filtered with filter paper No. 5A. The filtrate was passed through a membrane having a pore diameter of 0.8 μm to collect E. coli. And, 10 mL of sterile diluting water containing 0.04 vol. % Triton X-100 (Eiken Chemical Co., Ltd.) was twice suction-filtered to wash the filter. Furthermore, the filter after washing was inserted into a test tube and dipped in 2 mL of an enzyme substrate fluid (phosphate buffered saline [Nisshui Pharmaceutical Co., Ltd.] including 0.1 wt. % bovine serum albumin (BSA), 10 mM EDTA and, and 1 vol. % Glucuron (registered trademark) substrate fluid) to allow reaction to proceed at 37° C. with stirring. In this instance, the concentration of Triton X-100 was set at 0.1 to 0.8 vol. %. Ten minutes and 30 minutes after the reaction, 0.2 mL of the reaction solution was dispensed, and the same amount of a light emission accelerator [Accelerator II (trade name, Tropix, Inc.)] was added to each reaction fluid to measure the quantity of luminescence after two seconds. The measurement of the quantity of luminescence was performed using a luminometer (LB-9507, manufactured by Berthold Technology). The results are shown in FIG. 21. From the relationship between the enzyme activity and the concentration of Triton X-100, the concentration of Triton X-100 for obtaining a high enzyme activity was preferably higher and a preferred concentration of the surfactant was between 0.1 and 0.8 vol. %.

### Example 8

### Rapid/High Sensitivity Measurement of E. coli

It is shown that in the method of measuring β-glucuronidase activity according to the present invention, the combination of the concentration by a membrane filter and the washing of the membrane can render the measurement of E. coil quick and high sensitive.

First settling basin overflow and the secondary treatment water in a sewage treatment plant were collected as samples and immediately subjected to measurement. The number of E. coli by the incubation method (the colony counting method) was calculated by incubating the samples at 37° C. for 20 hours with the use of a Petrifilm (registered trademark) “E.coli/coliform measuring EC plate”, a product of 3M, and then allowing the incubated product to react with X-glu of an enzyme substrate specific to β-glucuronidase to form blue colonies and counting the gas producing blue colonies as E. coli. As a result, the number of E. coli in the first settling basin overflow was 9.1×10⁴ CFU/mL and that in the secondary treatment water was 2.3×10² CFU/mL.

First of all, the enzyme activity of this secondary treatment water was measured. Using filter paper No. 5A, 100 mL of the secondary treatment water was suction-filtered and the filtrate was filtered with a membrane filter made of a mixed cellulose ester having a pore diameter of 1.0 μm (diameter 25 mm) to collect bacteria on the filter. Also, sterile diluting water containing 0.04 vol. % Triton X-100 (Eiken Chemical Co., Ltd.) was twice suction-filtered to wash the filter. Further, the filter after washing was placed in a transparent glass vial having a diameter of 30 mm so as to render the filter surface below and was dipped in 1 mL of an enzyme substrate fluid (phosphate buffered saline (Nissui Pharmaceutical Co., Ltd.) containing 0.1 wt. % bovine serum albumin (BSA), 0.1 vol. % Triton X-100, 10 mM EDTA and 1 vol. % of Glucuron (registered trademark) substrate fluid) to allow reaction to proceed at 37° C. with stirring. 10 minutes, 20 minutes and 30 minutes after the enzyme reaction, 300 μL of respective reaction solution was collected, and 300 μL of a light emission accelerator [Accelerator II (trade name, Tropix, Inc.)] was added thereto to measure the quantity of luminescence after two seconds. The measurement of the quantity of luminescence was performed using a luminometer (LB-9507, manufactured by Berthold Tecnology). The relative light unit (RLU) versus the shaking time (enzyme substrate reaction time) in each sample was plotted on a graph and the gradient of the obtained straight line was taken as an enzyme activity (β-glucuronidase activity).

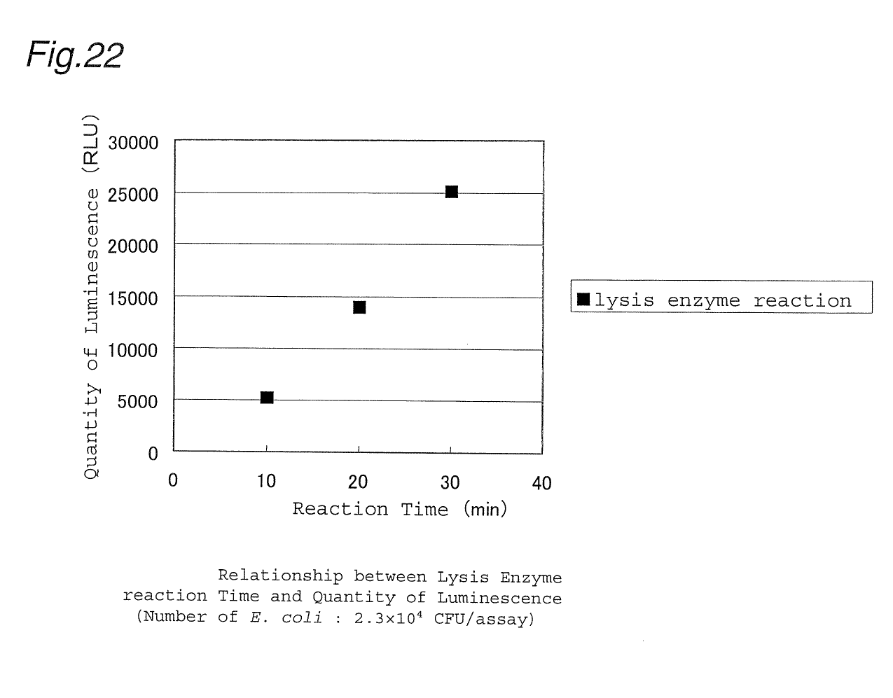

FIG. 22 shows one example of the graph of the relative light unit (RLU) versus the shaking time (enzyme substrate reaction time) as prepared in the above described manner. According to the graph of FIG. 22, the enzyme activity (the gradient of the straight line) was 996 RUL/min. Even in the case of allowing the lysis treatment and the enzyme reaction to simultaneously proceed, the quantity of luminescence increased in accordance with the reaction time.

Next, by adding 0.0001 to vol. % of the previous first settling basin overflow to E. coli-free advanced treatment water in a sewage treatment plant, a sample containing a predetermined concentration of E. coli was prepared, and the change in the quantity of luminescence from 10 minutes up to 30 minutes by the previously shown method (the same method as in FIG. 22) was taken as the β-glucuronidase activity.

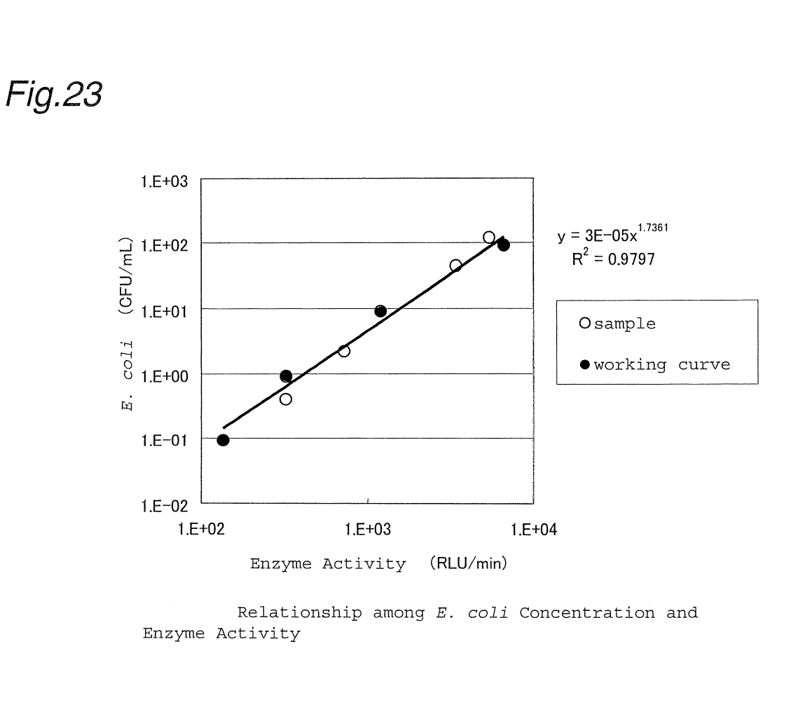

With the same sample, the number of E. coli was measured by the incubation method (colony counting method), and the correlation with this enzyme activity is as shown in FIG. 23.

As is shown in FIG. 23, the enzyme activity obtained a very good straight line relation in the range of the number of E. coli by the incubation method of 0.09 CFU/mL to 91 CFU/mL. Further, the lower limit of the quantification of E. coli was improved by 10,000 times or more from 1.0×10³ CFU/mL by the conventional method (the method disclosed in George, I., Crop, P., and Servais, P., “Use of β-D-galactosidase and β-D-glucuronidase activities for quantitative detection of total and fecal coliforms in wastewater”, Can., J. Microbiol., 47, 670-675 (2001)). Next, the enzyme activitys of two test samples of actual final settling basin overflow in a sewage treatment plant and one test sample of disinfection treated water were measured by the same procedure, and using this working curve, the concentration of E. coli was calculated. The results are shown in Table 3.

TABLE 3
Comparison of Measured Value of Number of E. coli
by Incubation with by This Invention
Number of E. coli (number/mL)

| Incubation Method | This Invention |
|---|---|
| 120 | 91 |
| 45 | 41 |
| 2.2 | 2.7 |

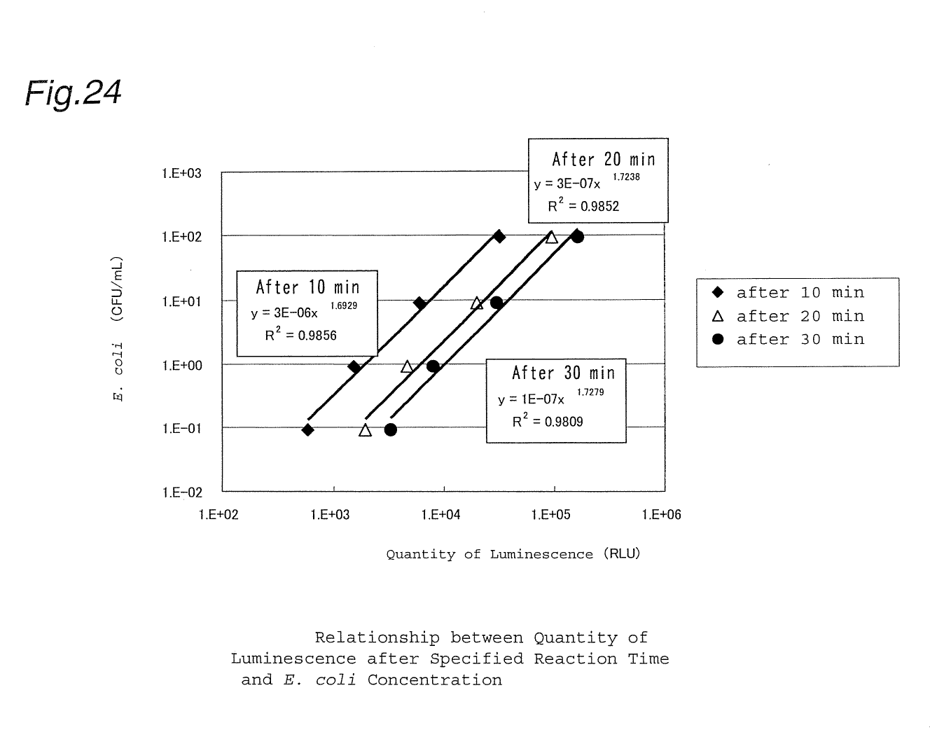

The concentration of E. coli measured according to the method of the present invention agreed with the number of E. coli measured according to the incubation method. Furthermore, the correlation between the quantities of luminescence 10 minutes, 20 minutes and 30 minutes after the lysis enzyme reaction of the sample used in the working curve of FIG. 23 and the numbers of E. coli by the incubation method was as shown in FIG. 24. Since the quantities of luminescence 10 to 30 minutes after the lysis enzyme reaction showed a high correlation with the concentration of E. coli as with the enzyme activity, it was found that the number of E. coli could be measured with the quantity of luminescence after 10 minutes.

### Example 9

Next, in order to more simply measure the number of E. coli, a technique of measuring the quantity of luminescence in the state of the filter dipped in an enzyme reaction vessel was examined.

To a vial in which the filter collected bacteria was dipped to allow enzyme reaction to proceed for 30 minutes in the same manner as in Example 8, 1 mL of a light emission accelerator [Accelerator II (trade name, Tropix, Inc.)] was directly added to measure the quantity of luminescence after 2 seconds. At this time, the measurement of quantity of light emission was performed with the use of a luminometer (LUMI COUNTER 2,500 A, manufactured by Microtec Co., Ltd.) capable of directly measuring a diameter of 30 mm vial) by photometry of the entire surface of the filter from under the vial. Further, 300 μL of the reaction fluid 30 minutes after the enzyme reaction was dispensed to a test tube and 300 μL of a fermentation accelerator was added thereto to measure the quantity of luminescence in the same manner.

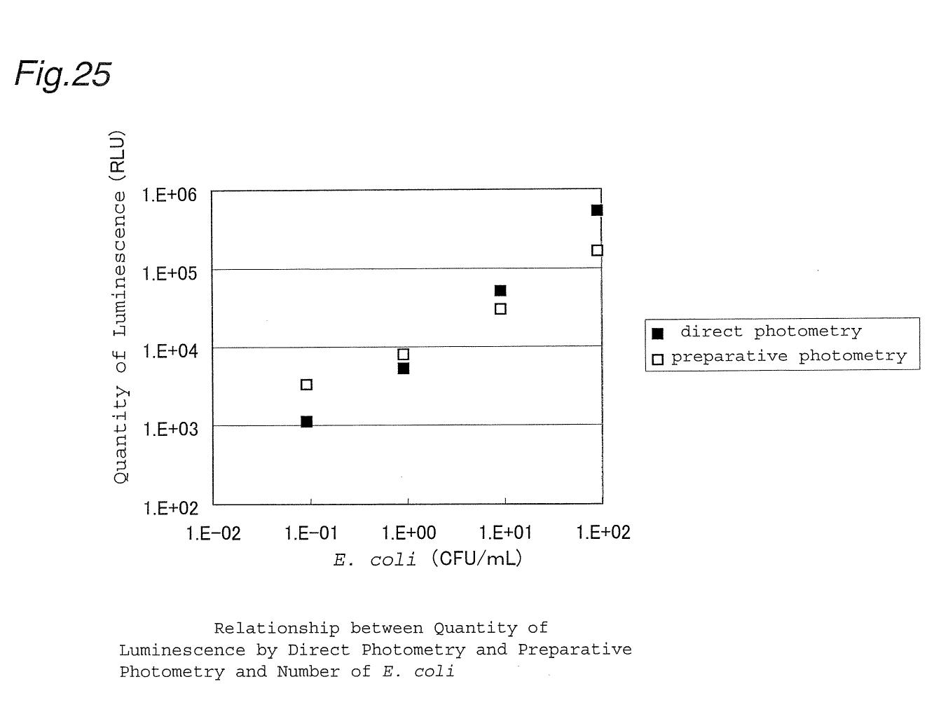

The results are shown in FIG. 25 and even the reaction fluid including the filter showed a high correlation with the number of bacterial according to the incubation method. Furthermore, it was found that on subjecting the above described sample to luminescence measurement, the quantity of luminescence on the high concentration side was further amplified to reduce the background as well due to the reflective effect of light by the filter surface, and thus it was possible to measure with higher accuracy. It has been impossible heretofore to measure a sample in which the filter disturbing the transmission of excitation light was dipped in the enzyme activity measurement by fluorescence method but by using the present luminescence measuring method it has become possible to directly measure the quantity of luminescence of the reaction fluid in the state of the filter dipped therein and simple measurement with high accuracy became possible.

### Example 10

### Proving Test with Treatment Water in Sewage Treatment Plant

Secondary treatment water and aerobic basin outlet water collected from a sewage treatment plant were dispersed in an ultrasonic bath and then suction-filtered with the use of filter paper No. 5A. The filtrate was passed through a membrane having a pore diameter of 0.8 μm to collect E. coli. As the enzyme extracting solution, a solution obtained by adding 0.2% Triton X-100 to a 0.1 M sodium phosphate solution containing 0.1 vol. % BSA was used and the E. coli collected membrane was dipped in this solution to perform lysis treatment at 37° C. for 30 minutes. One hundred microliters of the supernatant was collected and mixed with 180 μL of the above described enzyme reaction substrate solution. By the same procedure as that used in Example 2, the mixture was shaken at 37° C. for a predetermined time using a thermostatic shaker, and then 300 μL of a light emission accelerator was added thereto to cause luminescence and the quantity of luminescence was measured to find an enzyme activity. Separately, the true number of E. coli in the same sample was found by the incubation method (the colony counting method). Petrifilm (trademark) “E-coli/coliforms measuring EC plate”, a product of 3M was used as the medium. The sample was incubated at 37° C. for 20 hours, and thereafter allowed to react with X-glu of an enzyme substrate specific β-glucuronidase to form blue colonies, and the gas generating blue colonies were counted as E. coli.

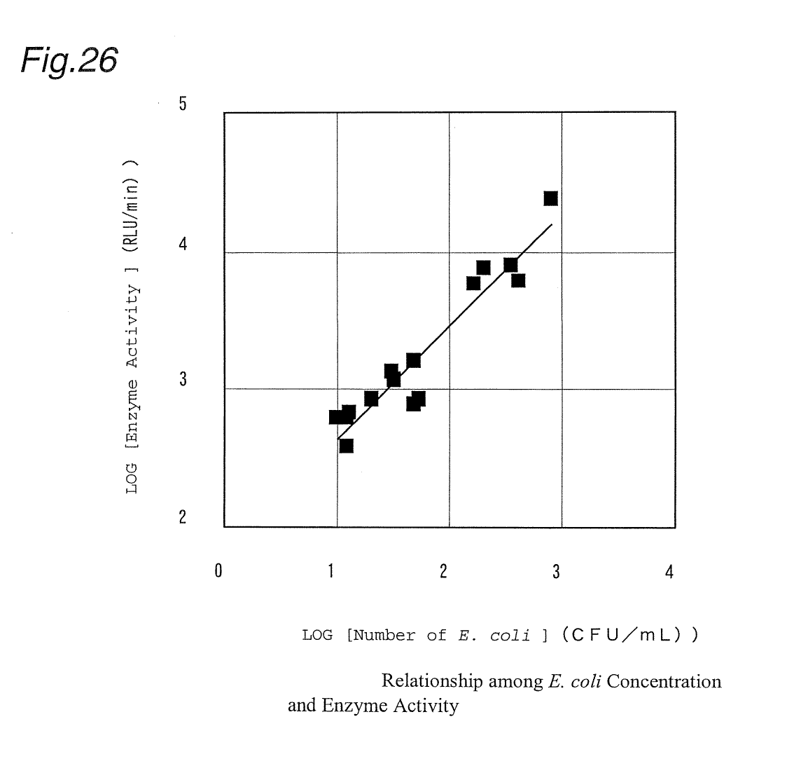

The correlation between the number of E. coli (colony count number) and the β-glucuronidase activity found by this technique is shown in FIG. 26.

As a result of statistically analyzing the results of FIG. 26, 95% confidence interval was merged into the range for practical purposes. The present invention is simple, quick and highly sensitive and additionally improves the measuring accuracy and, as a result, the number of bacteria in the range of from high concentration E. coli to low concentration E. coli can be accurately counted.

### Example 11

### Measurement of Number of Coliforms Based on β-Glucuronidase Activity

To secondary treatment water and aerobic basin outlet water in a sewage treatment plant, the method of this invention was applied. The test sample water was dispersed in an ultrasonic bath and then suction-filtered using filter paper No. 5A. The filtrate was passed through a membrane having a pore diameter of 0.8 μm to collect E. coli. The E. coli collected membrane was impregnated with an extracting solution containing 0.2 vol. % Triton X-100 to lyse E. coli at 37° C. for 30 minutes. One hundred microliters of the supernatant was collected and was mixed with 180 μL of a substrate solution comprising 1 vol. % Glucron (registered trademark), 0.1 M sodium phosphate and 10 mM EDTA. By the same procedure as in Example 2, the mixture was shaken at 37° C. for a predetermined time with the use of a thermostatic shaker, and then 300 μL of a light emission accelerator was added thereto to cause luminescence and the quantity of luminescence was measured to find an enzyme activity.

Separately, the true number of coliforms was found according to the incubation method (the colony counting method). Petrifilm (registered trademark) “E-coli/coliforms measuring EC plate”, a product of 3M was used as the medium. The sample was incubated at 37° C. for 20 hours, and thereafter allowed to react with X-gal of an enzyme substrate specific to β-galactosidase to form red colonies, and the gas-generating red colonies were counted as coliforms.

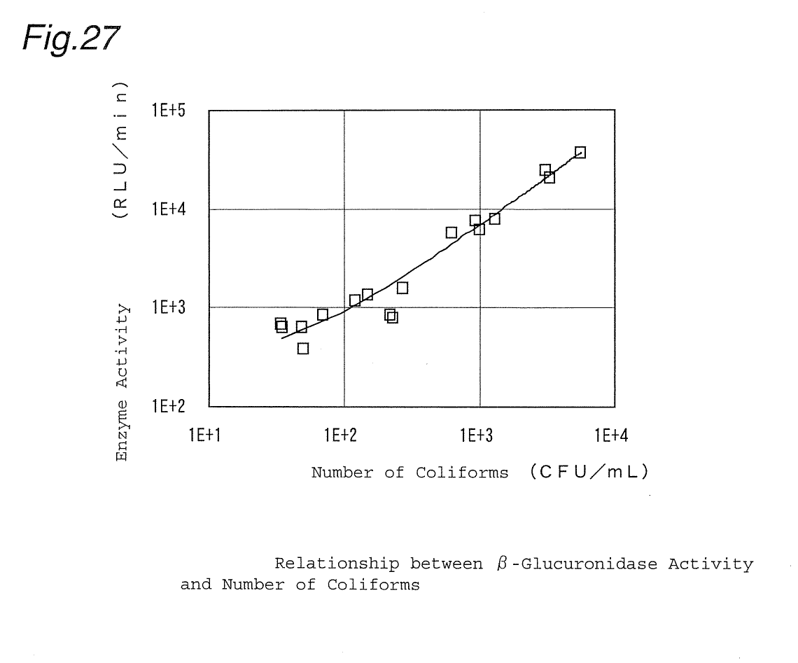

The relationship between the number of coliforms (colony count value) and the enzyme activity (β-glucuronidase activity) obtained by the above described technique is shown in FIG. 27.

From FIG. 27, it is understood that the β-glucuronidase activity and the number of coliforms are in a linear correlation. From this result, it has been proved that on measuring the β-glucuronidase activity, the calculation of coliforms is possible.

### Example 12

### Proving Test with Seawater

In a preferred embodiment of the present invention, the bacteria collected with a filter membrane have been washed with a surfactant, and accordingly the enzyme reaction is not susceptible to the inhibition by the composition of a solution. Thus, the present Example shows that according to a preferred embodiment, for example, even with a sample having a high concentration salinity, rapid/high sensitivity measurement is possible.

To seawater having a high E. coli concentration due to the effect of combined sewer overflow during wet weather, the method of this invention was applied. In an ultrasonic bath, 100 mL of the test sample water was dispersed and then suction-filtered with the use of filter paper No. 5A. The filtrate was passed through a membrane having a pore diameter of 1.0 μm to collect E. coli. By twice passing a solution containing 10 mL of 0.02 vol. % Triton X-100 through the membrane collected E. coli, filter washing was performed.

Next, the membrane was impregnated with an extracting solution containing 0.2 vol. % Triton X-100 to lyse the bacteria at 37° C. for 30 minutes. One hundred microliters of the supernatant was collected and was mixed with 180 μL of a substrate solution comprising 1 vol. % Glucuron (registered trademark) 0.1 M sodium phosphate and 10 mM EDTA. By the same procedure as in Example 2, the mixture was shaken at 37° C. for a predetermined time with using a thermostatic shaker, and then 300 μL of a light emission accelerator was added thereto to cause luminescence and the quantity of luminescence was measured to find an enzyme activity.

Separately, the true number of E. coli was found by the incubation method (the colony counting method). Petrifilm (registered trademark) “E-coli coliforms measuring EC plate”, a product of 3M was used as the medium. The sample was incubated at 37° C. for 20 hours, and thereafter allowed to react with X-glu of an enzyme substrate specific to β-glucuronidase to form blue colonies, and the gas generating blue colonies were counted as E. coli.

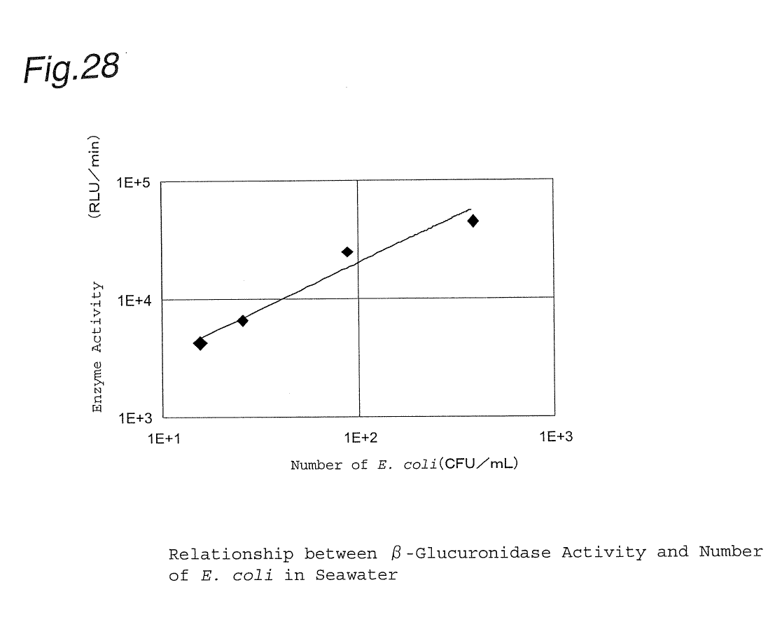

The correlation between the number of E. coli (colony count value) and the enzyme activity (β-glucuronidase activity) obtained by the above described technique is shown in FIG. 28.

From FIG. 28, it is understood that the measurement of the number of E. coli based on the β-glucuronidase activity is not affected by seawater and a linear correlation up to a low concentration of bacteria exists. From this result, it has been proved that according to the method relating to a preferred embodiment of the present invention, the enzyme reaction is not susceptible to the inhibition by the solution composition due to the washing of the bacteria collected with a filter membrane.

### Example 13

### Proving Test with Seawater

To seawater having a high E. coli concentration due to the effect of combined sewer overflow during wet weather, the method of this invention was applied. In an ultrasonic bath, the test sample water was dispersed and then suction-filtered with the use of filter paper No. 5A. The filtrate was passed through a nitrocellulose membrane having a pore diameter of 1.0 μm to collect E. coli. By twice passing 10 mL of a solution containing 0.02 vol. % Triton X-100 through the bacteria collected membrane filter, filter washing was performed. Next, the membrane was impregnated with an extracting solution containing 2 ml of a lysing enzyme substrate fluid (phosphate buffered saline (Nissui Seiyaku) containing 0.1 wt. % bovine serum albumin (BSA), 0.1 vol. % Triton X-100, 10 mM EDTA and, further 1 vol. % Glucuron (registered trademark) substrate fluid (a product of Applied Biosystem)) to allow enzyme reaction to proceed at 37° C. for 30 minutes. Ten minutes, 20 minutes and 30 minutes after the enzyme reaction, 300 μL of respective reaction solutions was collected and 300 μL of a light emission accelerator was added thereto to cause luminescence, and the quantity of luminescence after two seconds was measured to find an enzyme activity.

Separately, the true number of fecal coliforms was found by the incubation method (the colony counting method). With sterile HA Filter (a product of Millipore), 100 mL of the sample was filtered, and the resulting filter was placed in a laboratory dish containing a pad impregnated with the m-FC medium (m-FC Broth, a product of Millipore), and incubated at 44.5° C. for 22 hours. Also, the formed colonies were counted as the number of fecal coliforms.

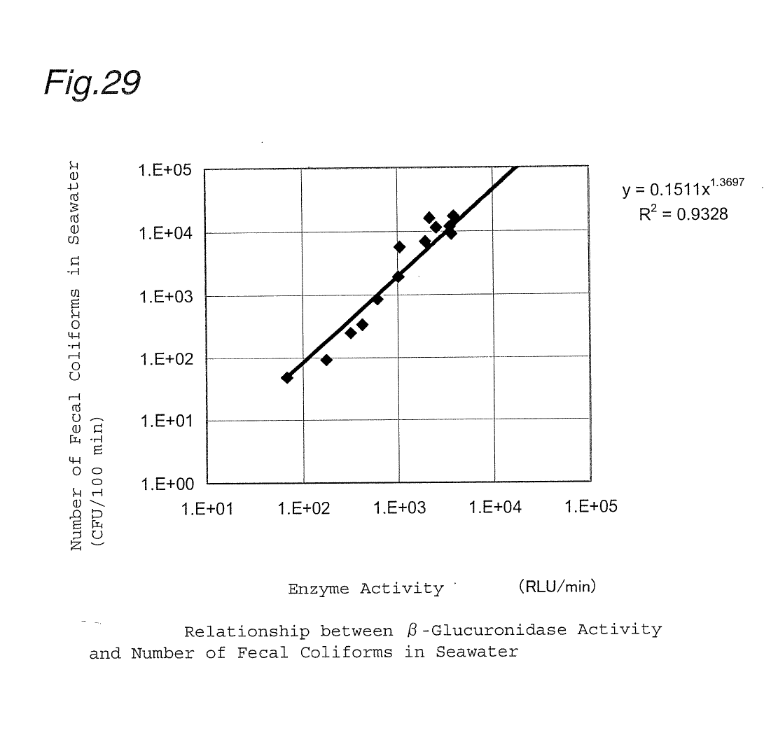

The correlation between the number of fecal coliforms (colony count value) and the enzyme activity (β-glucuronidase activity) obtained by the above described technique is shown in FIG. 29.

From FIG. 29, it is understood that the measurement of the number of fecal coliforms based on the β-glucuronidase activity is not affected by seawater and a linear correlation up to a low concentration exists. According to the present invention, in addition to simplicity, rapidness and high sensitivity, the measuring accuracy was improved and as a result, it became possible to accurately calculate the number of bacteria from low concentration E. coli to high concentration E. coli.

From this result, it has been proved that according to the method relating to a preferred embodiment of the present invention, the enzyme reaction is not susceptible to inhibition by the solution composition due to the washing of the bacteria collected with a filter membrane with a surfactant.

### INDUSTRIAL APPLICABILITY

E. coli is important as a hygienic indicator and various rapid measuring methods have been proposed. However, all these measuring methods have problems and could not be suitable for practical purposes. Compared to the conventional techniques (George, I., Crop, P., and Servais, P., “Use of β-D-galactosidase and β-D-glucuronidase activities for quantitative detection of total and fecal coliforms in wastewater”, Can., J. Microbiol., 47, 670-675 (2001)) having the same level of rapidness (a measuring time of about one hour), the method of the present invention has improved sensitivity by 40 times to 100 times or more. Further, compared to the conventional technique (Japanese Patent Publication A No. H11-505405) having the same level of sensitivity (1 CFU/mL), the measuring time has been shortened by 1/6.5 to 1/38. The present invention is a feasible measuring method for the first time as the calculation method of the hygienic indicator suitable for judging the discharging criterion value. The measurement of the number of bacteria by the conventional colony counting has required 24 hours. Accordingly, even when the number of bacteria is within the discharging criterion value, sterilization treatments such as chlorine addition, UV irradiation and ozone treatment have been carried out for the sake of safety. Further, according to the conventional methods, the measuring accuracy is low, and thus the measuring result could not be used as a hygienic indicator and safety could not be judged. On account of this, these sterilization treatments could not be omitted. In recent years, it is pointed out that by-products with chlorine treatment and ozone treatment harm the environment. Further, UV irradiation and ozone treatment require large energy in the treatments. By rapidly measuring E. coli/coliforms using the present invention, the sterilization treatment may be carried out only when required, and thus the resulting environmental protection and energy-saving is highly significant.

Further, the present invention can be utilized in not only the property analysis of sewage but also the property analysis (measurement of the number of E. coli and coliforms) of tap water such as drinking water.

What is claimed is:

1. A method of detecting bacteria comprising passing a sample fluid through a filter membrane having a pore diameter of 0.6 to 5.0 μm and/or a filter membrane having a flow rate of distilled water passed, measured in accordance with ASTM F317-72, of 50 to 500 mL/min·cm² to collect the bacteria in the sample fluid on the filter membrane, bringing the filter membrane into contact with a lysing agent and an enzyme reaction substrate and finding an enzyme activity of an enzyme in the target bacteria to the enzyme reaction substrate to quantify the number of bacteria in the sample liquid.

2. The method of claim 1, wherein said filter membrane is a nitrocellulose membrane having a pore diameter of 0.8 to 3.0 μm.

3. The method of claim 1 comprising passing a surfactant through the filter membrane to wash the filter membrane before bringing the filter membrane into contact with the lysing agent and the enzyme reaction substrate.

4. The method of claim 1, wherein the filter membrane is brought into contact with the lysing agent and the enzyme reaction substrate simultaneously.

5. The method of claim 1, wherein the filter membrane is brought into contact with the lysing agent to extract an enzyme before the extracted enzyme is brought into contact with the enzyme reaction substrate.

6. The method of claim 1, wherein the bacteria of the target for measurement are E. coli, coliforms and/or fecal coliforms, and the number of E. coli, coliforms and/or fecal coliforms in the sample fluid is quantified based on the enzyme activity of β-glucuronidase to be extracted from E. coli, coliforms and/or fecal coliforms.

7. The method of claim 6, wherein 3-(4-methoxyspiro{1,2-dioxetane-3,2′-(5′-chloro)-tricyclo-[3.3.1.1^{3,7} ]decan}-4-yl)phenyl-β-D-glucronide or its derivative is used as the enzyme reaction substrate.

8. The method of claim 1 further comprising adding a light emission accelerator and measuring the quantity of chemiluminescence of a luminescence to determine the enzyme activity of the enzyme in the target bacteria to the enzyme reaction substrate.

9. An apparatus for detecting bacteria comprising:
a collection vessel comprising a filter membrane having a pore diameter of 0.6 to 5.0 μm and/or a filter membrane having a flow rate of distilled water passed, measured in accordance with ASTM F317-72, of 50 to 500 mL/min·cm²;
a sample fluid feed mechanism for feeding a sample fluid to the primary side of the filter membrane;
an enzyme reaction substrate feed mechanism for feeding an enzyme reaction substrate; and
an enzyme activity measuring device for measuring an enzyme activity of an enzyme in the target bacteria to the enzyme reaction substrate.

10. The apparatus of claim 9, further comprising a light emission accelerator feed device for feeding an accelerator.

11. The apparatus of claim 9, wherein said filter membrane is a nitrocellulose membrane having a pore diameter of 0.8 to 3.0 μm.

12. The apparatus of claim 9, wherein said enzyme reaction substrate feed mechanism feeds a lysing agent in addition to the enzyme reaction substrate.

13. The apparatus of claim 9, further comprising a lysing agent feed device for feeding the lysing agent to the collection device.

14. An apparatus for detecting bacteria in a sample fluid comprising a collection unit for collecting the bacteria from the sample fluid, a reaction unit to allow enzyme substrate reaction to proceed and a means for transferring an enzyme extracted fluid from the collection unit to the reaction unit,
wherein said collection unit comprises:
a filter membrane having a pore diameter of 0.6 to 5.0 μm and/or a filter membrane having a flow rate of distilled water passed, measured in accordance with ASTM F317-72, of 50 to 500 mL/min·cm²;
a sample fluid feed device for feeding the sample fluid to a primary side of the filter membrane;
a washing solution feed device for feeding washing solution to the primary side of the filter membrane; and
a means for extracting an enzyme from the bacteria collected on the filter membrane; and
wherein said reaction unit comprises:
a reaction vessel for receiving the enzyme extracted fluid to allow enzyme substrate reaction;
an enzyme reaction substrate adding device for feeding an enzyme reaction substrate to the enzyme extracted fluid in the reaction vessel;
an accelerator adding device for adding an accelerator to the enzyme reaction substrate added reaction fluid in the reaction vessel; and
a measuring device for measuring the quantity of luminescence, fluorescence or color developing of the accelerator added reaction fluid.

15. The apparatus of claim 14, wherein said filter membrane is a nitrocellulose membrane having a pore diameter of 0.8 to 3.0 μm.

16. An apparatus for detecting bacteria in a sample fluid comprising a collection/reaction unit, a measuring unit and a means for transferring a reaction fluid after enzyme reaction from the collection/reaction unit to the measuring unit,
wherein said collection/reaction unit comprises:
a collection and reaction vessel for housing a filter membrane having a pore diameter of 0.6 to 5.0 μm and/or a filter membrane having a flow rate of distilled water passed, measured in accordance with ASTM F317-72, of 50 to 500 mL/min·cm²;
a sample feed device for feeding the sample fluid to a primary side of the filter membrane;
a washing solution feed device for feeding a washing solution to the primary side of the filter membrane;
a lysing enzyme reaction substrate adding device for feeding a lysing enzyme reaction substrate to the primary side of the filter membrane; and
a thermostatic device for maintaining the lysing enzyme reaction substrate fluid in the state of the filter membrane dipped therein at a constant temperature, and
wherein said measuring unit comprises:
a measuring vessel for receiving the reaction fluid after enzyme reaction;
an accelerator adding device for adding an accelerator to the reaction fluid after enzyme reaction in the measuring vessel; and
a measuring device for measuring the quantity of luminescence, fluorescence or color development of the accelerator added reaction fluid.

17. The apparatus of claim 16, wherein said filter membrane is a nitrocellulose membrane having a pore diameter of 0.8 to 3.0 μm.

18. The apparatus of claim 9 or 16, wherein the target bacteria are E. coli, coliforms and/or fecal coliforms.

19. A kit for simplified measurement of bacteria comprising:
a collection/reaction vessel equipped with a feed inlet and a discharge outlet comprising a filter membrane having a pore diameter of 0.6 to 5.0 μm and/or a filter membrane having a flow rate of distilled water passed, measured in accordance with ASTM F317-72, of 50 to 500 mL/min·cm²;
an enzyme reaction substrate fluid feed unit; and
an enzyme activity measuring device for measuring an enzyme activity.

20. The kit for simplified measurement of bacteria of claim 19, further comprising a washing solution feed unit and/or a light emission accelerator feed unit.

21. The kit for simplified measurement of bacteria of claim 19, further comprising a lysing agent feed unit.

22. The kit for simplified measurement of bacteria of claim 19, wherein said enzyme reaction substrate fluid feed unit contains a lysing agent and an enzyme reaction substrate fluid.

23. The kit for simplified measurement of bacteria of claim 19, wherein said filter membrane is a nitrocellulose membrane having a pore diameter of 0.8 to 3.0 μm.

24. The kit for simplified measurement of bacteria of claim 19, wherein the target bacteria are E. coli, coliforms fecal and/or coliforms.
